(19) United States
(12) Patent Application Publication
(10) Pub. No.: US 2023/0416707 A1
(43) Pub. Date: Dec. 28, 2023

(54) LONG-ACTING DNASE

(71) Applicant: Protalix Ltd., Carmiel (IL)

(72) Inventors: Ilya RUDERFER, Carmiel (IL); Liat FUX, Kiryat-Motzkin (IL); Yael HAYON, Tel Aviv (IL)

(73) Assignee: Protalix Ltd., Carmiel (IL)

(21) Appl. No.: 18/030,291

(22) PCT Filed: Oct. 7, 2021
(86) PCT No.: PCT/IL2021/051207

§ 371 Date: Apr. 5, 2023

Related U.S. Application Data
(60) Provisional application No. 63/088,496, filed on Oct. 7, 2020.

(51) Int. Cl. C12N 9/22; A61K 47/60; A61P 1/00; A61P 7/00; A61P 31/04; A61P 9/10; A61P 31/12

(52) U.S. Cl. CPC C12N 9/22; A61K 47/60; A61P 1/00; A61P 7/00; A61P 31/04; A61P 9/10; A61P 31/12; C12Y 301/21001

(57) ABSTRACT

A modified DNase protein is described herein as well as pharmaceutical compositions comprising same, the modified DNase protein comprising a DNase polypeptide attached to at least two poly(alkylene glycol) moieties. Further described herein is a process of preparing a modified DNase protein, the process comprising: contacting the polypeptide with an agent that comprises a poly(alkylene glycol) attached to an aldehyde group, to obtain a conjugate of the polypeptide and the agent; and contacting the conjugate with a reducing agent.

### RELATED APPLICATION

This application claims the benefit of priority of U.S. Provisional Patent Application No. 63/088,496 filed on Oct. 7, 2020, the contents of which are incorporated herein by reference in their entirety.

### SEQUENCE LISTING STATEMENT

The ASCII file, entitled 89420.txt, created on 5 Oct. 2021, comprising 16,384 bytes, submitted concurrently with the filing of this application is incorporated herein by reference.

### FIELD AND BACKGROUND OF THE INVENTION

The present invention, in some embodiments thereof, relates to therapy, and more particularly, but not exclusively, to long-acting DNase.

Based on their biochemical properties and enzymatic activities deoxyribonuclease (DNase) proteins have been classified as two types, DNase I and DNase II. DNase I proteins have a pH optimum near neutrality, and produce 5′-phosphate nucleotides upon hydrolysis of DNA.

Human DNase I is a member of the mammalian DNase I family (EC 3.1.21.1). DNase I belongs to the class of Mg²⁺ and Ca²⁺ dependent endonucleases, whose hydrolytic activity depends on the presence of divalent cations. Mg²⁺ ion is involved in electrophilic catalysis of the phosphodiester bond cleavage, whereas Ca²⁺ maintains optimal enzyme conformation. DNase I cleaves DNA preferentially at phosphodiester linkages adjacent to a pyrimidine nucleotide, yielding 5′-phosphate-terminated polynucleotides with a free hydroxyl group on position 3′, on average producing tetranucleotides. DNase I acts on single-stranded DNA, double-stranded DNA, and chromatin.

DNase II (EC 3.1.22.1) cleaves DNA preferentially at phosphodiester linkages so as to yield products with 3′-phosphates and 5′-hydroxyl ends. DNase II functions optimally at acidic pH, and is commonly found in lysosomes.

The principal therapeutic use of human DNase has been to reduce the viscoelasticity of pulmonary secretions (including mucus) in diseases such as pneumonia and cystic fibrosis (CF), by hydrolyzing high molecular weight DNA that is present in such secretions, thereby aiding in the clearing of respiratory airways [Shak et al., PNAS 87:9188-9192 (1990)]. Mucus also contributes to the morbidity of chronic bronchitis, asthmatic bronchitis, bronchiectasis, emphysema, acute and chronic sinusitis, and even the common cold. The pulmonary secretions of persons having such diseases are complex materials that include mucus glycoproteins, mucopolysaccharides, proteases, actin, and DNA. DNase has also been proposed for non-pulmonary disorders, for example, treatment of male infertility and uterine disorders (see U.S. Patent Application Publication No. 2007/0259367), inhibition of metastatic growth (see U.S. Pat. No. 7,612,032) and topical application for diabetic wound healing.

Dornase alfa is a recombinant human DNase (rhDNase) expressed in Chinese hamster ovary (CHO) cells, used in the treatment of cystic fibrosis, and marketed under the trade name Pulmozyme®.

International Patent Application Publication WO 2013/114374 describes plant-expressed human recombinant DNase I proteins, and uses thereof for treating pulmonary and/or respiratory conditions by inhalation of the DNase I.

International Patent Application Publication WO 2016/108244 describes modified DNase I protein which exhibits an improved DNA hydrolytic activity compared to a homologous non-modified DNase I protein. An exemplary modified DNase I protein which has undergone clinical trials is referred to as “alidornase alfa”.

Dwyer et al. [J Biol Chem 1999, 274:9738-9743] describes expression and purification of a DNase I-Fc fusion protein, resulting in a dimeric form of DNase I. The dimeric DNase I-Fc fusion protein was functionally active in enzymatic DNA digestion assays, albeit about 10-fold less than monomeric DNase I.

International Patent Application Publication WO 2015/107176 describes PEGylation of a therapeutic agent for treating a respiratory disease with one or more PEG moiety having a molecular weight of more than 12 kDa. In particular, dornase alfa with one PEG moiety (20-40 kDa) conjugated to the N-terminus thereof is described.

Russian Patent No. 2502803 describes introduction of cysteine residues into DNase for conjugation with 10 kDa PEG-maleimide, resulting in PEGylated DNase which exhibits 10-20% of the enzymatic activity of non-modified DNase.

Patel et al. [Appl Nanosci 2020, 10:563-575] describes DNase-I functionalized chitosan nanoparticles loaded with ciprofloxacin for preventing Pseudomonas aeruginosa biofilm development.

Park et al. [Sci Transl Med 2016, 8:361ra131] describes nanoparticles coated with DNase I for inhibiting metastasis.

Meng et al. [Recent Pat Drug Deliv Formul 2018, 12:212-222] describes polysialylation of DNase I or erythropoietin in order to improve stability against proteases and thermal stress, with slightly reduced enzymatic activity.

U.S. Pat. No. 7,846,445 describes an unstructured recombinant polymer (URP) comprising at least 40 contiguous amino acids, which may lengthen a serum excretion half-life and/or increase solubility of a protein into which the URP is incorporated.

XL-protein GmbH reported the generation of recombinant human DNase I with prolonged half-life by incorporating a disordered polypeptide chain at an N-terminus of the DNase [www(dot)rentschler-biopharma(dot)com/fileadmin/user_upload/Scientific-Posters/Rentschler_Poster_ESACT_2019_PASylated_human_DNase_I_final_screen].

Neutrophil extracellular traps (NETs) are networks of extracellular fibers, primarily composed of DNA from neutrophils, which bind pathogens. NET activation and release (also known as “NETosis”) may involve neutrophil death (suicidal NETosis) or exocytosis which does not result in neutrophil death (vital NETosis). NETs may contribute to innate immunity by binding to microorganisms and preventing dissemination of pathogens, but result in increased thrombosis and damage of endothelium and other tissue [Mai et al., Shock 2015, 44:166-172; McDonald et al., Cell Host Microbe 2012, 12:324-333; Papayannopoulos, Nat Rev Immunol 2018, 18:134-147].

Czaikoski et al. [PLoS One 2016, 11:e0148142] reported that systemic recombinant human DNase treatment reduces serum NETs and increased bacterial load in septic mice; whereas DNase treatment plus antibiotics attenuated organ damage and improved survival rate.

U.S. Patent Application Publication No. 2020/0024585 describes engineered DNase proteins for treating conditions characterized by neutrophil extracellular trap (NET) accumulation and/or release, such as vascular occlusions involving NETs.

Additional background art includes Dwivedi et al. [Crit Care 2012; 16:R151]; Ehrlich et al. [J Mol Recognition 2009, 22:99-103]; Garay et al. [Expert Opin Drug Deliv 2012, 9:1319-1323]; Guichard et al. [Clin Sci (Lond) 2018, 132:1439-1452]; Lubich et al. [Pharm Res 2016, 33:2239-2249]; Moreno et al. [Cell Chem Biol 2019, 26:634-644]; Pressler [Biologics 2008, 2:611-617]; Rudmann et al. [Toxicologic Pathology 2013, 41:970-983]; Wan et al. [Process Biochemistry 2017, 52:183-191]; and Zhang et al. [J Control Release 2016, 244:184-193]; and U.S. Pat. Nos. 8,431,123, 8,871,200, 8,916,151, 9,642,822, and 9,770,492.

### SUMMARY OF THE INVENTION

According to an aspect of some embodiments of the invention, there is provided a modified DNase protein comprising a DNase polypeptide attached to at least two poly(alkylene glycol) moieties.

According to an aspect of some embodiments of the invention, there is provided a pharmaceutical composition comprising a modified DNase protein according to any of the respective embodiments described herein, and a pharmaceutically acceptable carrier.

According to an aspect of some embodiments of the invention, there is provided a process of preparing a modified DNase protein according to any of the respective embodiments described herein, the process comprising:
(a) contacting the polypeptide with an agent that comprises a poly(alkylene glycol) attached to an aldehyde group, to obtain a conjugate of the polypeptide and the agent; and
(b) contacting the conjugate with a reducing agent.

According to some of any of the embodiments of the invention, at least a portion, or each, of the at least two poly(alkylene glycol) moieties has a molecular weight of no more than about kDa, optionally no more than about 7.5 kDa, and optionally no more than about 5 kDa.

According to some of any of the embodiments of the invention, at least a portion, or each, of the at least two poly(alkylene glycol) moieties has a molecular weight in a range of about 2 kDa to about 5 kDa.

According to some of any of the embodiments of the invention, the polypeptide is attached to from 2 to 7 poly(alkylene glycol) moieties.

According to some of any of the embodiments of the invention, the polypeptide is attached to at least three poly(alkylene glycol) moieties, optionally from 3 to 6 poly(alkylene glycol) moieties.

According to some of any of the embodiments of the invention, the polypeptide is attached to at least four poly(alkylene glycol) moieties, optionally from 4 to 6 poly(alkylene glycol) moieties.

According to some of any of the embodiments of the invention, at least a portion, or each, of the poly(alkylene glycol) moieties are monofunctional poly(alkylene glycol) moieties.

According to some of any of the embodiments of the invention, at least a portion, or each, of the poly(alkylene glycol) moieties comprise an alkylene group covalently attached to a nitrogen atom of an amine group in the polypeptide.

According to some of any of the respective embodiments of the invention, the amine group is comprised by a lysine residue side chain and/or the N-terminus.

According to some of any of the embodiments of the invention, at least 80%, and optionally about 100%, of the amine groups comprised by a lysine residue side chain and the N-terminus in the polypeptide are covalently attached to the poly(alkylene glycol) moieties.

According to some of any of the embodiments of the invention, at least a portion, or each, of the poly(alkylene glycol) moieties have formula I:

-L₂-L₁-[O—(CH₂)m]n-O—R₁ Formula I
wherein:
L₁ and L₂ are each independently a hydrocarbon moiety or absent;
R₁ is hydrogen or a hydrocarbon moiety;
m is an integer in a range of from 2 to 10; and
n is an integer in a range of from 2 to 1000.

According to some of any of the embodiments of the invention, at least a portion, or each, of the poly(alkylene glycol) moieties have formula I′:

—CH₂-L₁-[O—(CH₂)m]n-O—R₁ Formula I′
wherein:
L₁ is a hydrocarbon moiety or absent;
R₁ is hydrogen or a hydrocarbon moiety;
m is an integer in a range of from 2 to 10; and
n is an integer in a range of from 2 to 1000.

According to some of any of the respective embodiments of the invention, n is in a range of from 20 to 200, optionally from 30 to 150.

According to some of any of the respective embodiments of the invention, L₁ is an unsubstituted alkylene.

According to some of any of the respective embodiments of the invention, L₁ is from 1 to 6 carbon atoms in length, optionally two carbon atoms in length.

According to some of any of the embodiments of the invention, at least a portion, or each, of the poly(alkylene glycol) moieties are polyethylene glycol moieties.

According to some of any of the embodiments of the invention, the polypeptide is a recombinant polypeptide.

According to some of any of the embodiments of the invention, the polypeptide is a plant recombinant polypeptide.

According to some of any of the embodiments of the invention, the DNase protein is a DNase I protein.

According to some of any of the respective embodiments of the invention, the DNase I protein has at least 80% homology to a human DNase I protein.

According to some of any of the respective embodiments of the invention, the DNase I protein comprises or has the amino acid sequence as set forth in SEQ ID NO: 2.

According to some of any of the respective embodiments of the invention, DNase I protein comprises or has the amino acid sequence as set forth in SEQ ID NO: 1.

According to some of any of the respective embodiments of the invention, the composition or modified DNase protein according to any of the respective embodiments described herein is for use in treating a disease or disorder in which DNase activity is beneficial.

According to some of any of the respective embodiments of the invention, the composition or modified DNase protein according to any of the respective embodiments described herein is for use in treating a disease or disorder associated with excess extracellular DNA in a fluid, secretion or tissue of a subject in need thereof.

According to some of any of the respective embodiments of the invention, the disease or disorder is associated with neutrophil extracellular traps (NETs).

According to some of any of the respective embodiments of the invention, the disease or disorder is selected from the group consisting of thrombosis, vascular occlusion, an inflammatory disease or disorder, an autoimmune disease or disorder, a bronchopulmonary disease, a cardiovascular disease, a metabolic disease, a cancer, a neurodegenerative disease or disorder, a disease or disorder associated with an infection, liver damage, fibrosis, and a ductal occlusion.

According to some of any of the respective embodiments of the invention, the disease or disorder is selected from the group consisting of acute coronary syndrome, acute kidney injury, acute lung injury, acute respiratory distress syndrome, allergies, Alzheimer's disease, amyotrophic lateral sclerosis, arthritis, asthma, atelectasis, atherosclerosis, atopic dermatitis, bipolar disorder, bronchiectasis, bronchiolitis, bronchitis and tracheobronchitis, cholangitis, chronic kidney disease, chronic neutrophilia, chronic obstructive pulmonary disease, chronic suppurative lung disease conjunctivitis, common cold, cystic fibrosis, deep vein thrombosis, diabetes, disseminated intravascular coagulation, dry eye disease, empyema, endocarditis, female infertility, gout, graft-versus-host disease, hematomas, hemothorax, heparin-induced thrombocytopenia, hepatorenal syndrome, Huntington's disease, inflammatory bowel disease, intrabiliary blood clots, ischemia-reperfusion injury, Kartegener's syndrome, leukemia, leukostasis, liver cirrhosis, lupus nephritis, male infertility, mastitis, myocardial infarction, neutropenia, neutrophil aggregation, obstruction of the vas deferens, pancreatitis, Parkinson's disease, pneumonia, post-pneumatic anemia, primary ciliary dyskinesia, psoriasis, rhabdomyolysis, sarcoidosis, schizophrenia, sepsis, sickle cell disease, sinusitis, Sjogren's syndrome, smoke-induced lung injury, solid tumors and/or tumor metastasis, stroke, surgical adhesions, surgical and/or traumatic tissue injury, systemic inflammatory response syndrome, systemic lupus erythematosus, systemic sclerosis, thrombotic microangiopathy, tissue damage associated with irradiation and/or chemotherapy treatment, transfusion-induced lung injury, tuberculosis, vasculitis, venous thromboembolism, a viral, bacterial, fungal and/or protozoal infection, and a wound or ulcer.

According to some of any of the respective embodiments of the invention, the disease or disorder is sepsis.

According to some of any of the embodiments of the invention relating to a process, the reducing agent is selected from the group consisting of a picoline borane complex and a cyanoborohydride.

According to some of any of the embodiments of the invention relating to a process, the agent has formula II:

HC(═O)-L₁-[O—(CH₂)m]n-O—R₁ Formula II
wherein:
L₁ is a hydrocarbon moiety;
R₁ is hydrogen or a hydrocarbon moiety;
m is an integer in a range of from 2 to 10; and
n is an integer in a range of from 2 to 1000.

According to some of any of the embodiments of the invention relating to a process, a molar ratio of the agent to the polypeptide is in a range of from 10:1 to 2,000:1.

According to some of any of the embodiments of the invention relating to a process, contacting the conjugate with a reducing agent is effected at a pH of at least about 7.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents an image of an SDS-PAGE (Tris acetate 3-8%) gel with DNase I before modification (BM) and upon modification with PEG-Ald (5 kDa) obtained from CreativePEGWorks (1), NOF Europe (2), and JenKem Technology (3), or with PEG-NHS (5 kDa) obtained from Rapp Polymere (4) and Iris Biotech (5) (molecular weight indicators are provided in lane M).

FIG. 2 presents an image of an SDS-PAGE (Tris acetate 3-8%) gel with DNase I before modification (BM) and upon modification by 200 (1), 400 (2) and 600 (3) equivalents of PEG-Ald (2000 Da), or by 200 (4), 400 (5) and 600 (6) equivalents PEG-Ald (5000 Da) respectively.

FIG. 3 presents an image of an SDS-PAGE gel with plant recombinant (pr) human DNase I before modification (BM) and upon PEGylation by PEG-Ald (5000 Da) (molecular weight indicators are provided in right lane).

FIG. 4 presents a graph showing DNase activity (average of 5 animals per data point) measured in plasma as a function of time following intravenous injection in rats of 1 mg/kg of exemplary DNase I modified with 5000 Da PEG, as determined by methyl green activity assay (also shown is best fit to data points and associated formula and R² values).

FIG. 5 presents an SDS-PAGE (Tris Acetate 3-8%) analysis of prh-DNase-1 before modification and prhDNase I modified with 2000 Da PEG, using 400 equivalents of PEG-Ald, or with 5000 Da PEG, using 100 (low) or 200 (high) equivalents of PEG-Ald.

FIGS. 6A and 6B present graphs showing (at different time scales) the concentration of DNase I in plasma as a function of time following intravenous injection in rats of 1 mg/kg of prhDNase I (plant recombinant human DNase I) modified with 2000 Da PEG or 5000 Da PEG (FIGS. 6A and 6B), or of non-modified prhDNase I or alidornase alfa (FIG. 6B), as determined by methyl green activity assay (“high” denotes about 4 PEG moieties per protein, “low” denotes about 3 PEG moieties per protein).

FIG. 7 presents a bar graph showing the half-life of DNase I in plasma following intravenous injection in rats of 1 mg/kg of prhDNase I modified with 2000 Da PEG or 5000 Da PEG or of non-modified prhDNase I or alidornase alfa (“high” denotes about 4 PEG moieties per protein, “low” denotes about 3 PEG moieties per protein).

FIG. 8 presents a bar graph showing the AUC (area under curve) of DNase I in plasma following intravenous injection in rats of 1 mg/kg of prhDNase I modified with 2000 Da PEG or 5000 Da PEG or of non-modified prhDNase I or alidornase alfa (“high” denotes about 4 PEG moieties per protein, “low” denotes about 3 PEG moieties per protein).

FIGS. 9A and 9B present mortality curves (FIG. 9A) and a bar graph showing average time until death (FIG. 9B) for mice subjected to CLP (cecal ligation and puncture) and treated with 10 mg/kg of long-acting (LA) prhDNase I modified with 5000 Da PEG 1 hour (3S) or 4 hours (4S) after CLP, 10 mg/kg of non-modified prhDNase I 1 hour (1S) or 4 hours (4S) after CLP, or saline (5S); statistical analysis by applying nonparametric comparison for each pair using Wilcoxon method (*p≤0.05, **p≤0.01; n=5 in each group).

FIGS. 10A and 10B present mortality curves (FIG. 10A) and a bar graph showing average time until death (FIG. 10B) for mice subjected to CLP and treated with 10 mg/kg of long-acting (LA) prhDNase I modified with 5000 Da PEG, or with saline, 4 hours after CLP; statistical analysis by applying nonparametric comparison for each pair using Wilcoxon method (*p≤n=5 in each group; for calculations, the two mice still alive after 7 days were considered to have died after 7 days).

FIGS. 11A and 11B present mortality curves (FIG. 11A) and a bar graph showing average time until death (FIG. 11B) for mice subjected to CLP and treated with 10 mg/kg of long-acting (LA) prhDNase I modified with 5000 Da PEG, or with saline, 8 hours after CLP; statistical analysis by applying nonparametric comparison for each pair using Wilcoxon method (*p≤n=3-5 in each group; for calculations, the four mice still alive after 7 days were considered to have died after 7 days).

FIGS. 12A and 12B present mortality curves (FIG. 12A) and a bar graph showing average time until death (FIG. 12B) for mice subjected to CLP and treated with 0.1, 1, 5 or 10 mg/kg of long-acting (LA) prhDNase I modified with 5000 Da PEG, or with saline, 4 hours after CLP; statistical analysis by applying nonparametric comparison for each pair using Wilcoxon method (*p≤0.05, **p≤0.01; n=5 in each group; for calculations, the mice still alive after 7 days were considered to have died after 7 days).

### DESCRIPTION OF SPECIFIC EMBODIMENTS OF THE INVENTION

The present invention, in some embodiments thereof, relates to therapy, and more particularly, but not exclusively, to long-acting DNase.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered that modification of a DNase protein with multiple poly(alkylene glycol) moieties can enhance the in vivo half-life, and consequently efficacy, of DNase to a considerable degree, and that the half-life can be readily controlled by controlling the degree of modification.

While reducing the invention to practice, the inventors have shown that exemplary modified DNase comprising polyethylene glycol of 2-5 kDa is more enzymatically active when modified by reductive amination than by amidation, that reductive amination is more effective than amidation for effecting modification, and that the modified DNase is therapeutically effective in a model of sepsis.

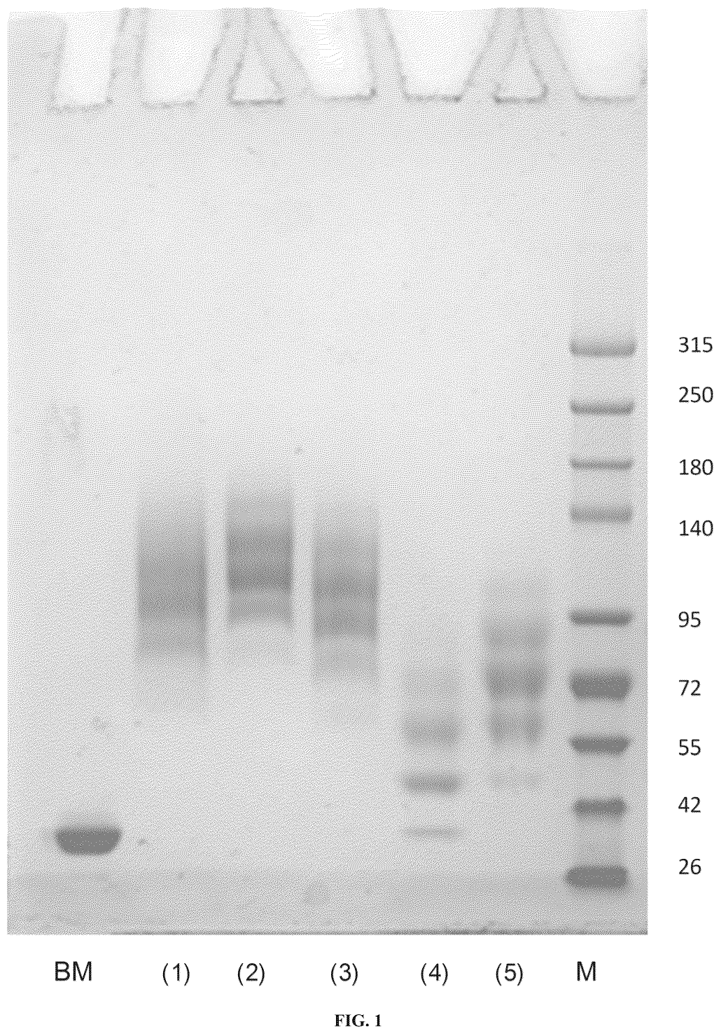

Referring now to the drawings, FIGS. 1-3 show the preparation of exemplary DNase proteins modified to various degrees by polyethylene glycol moieties.

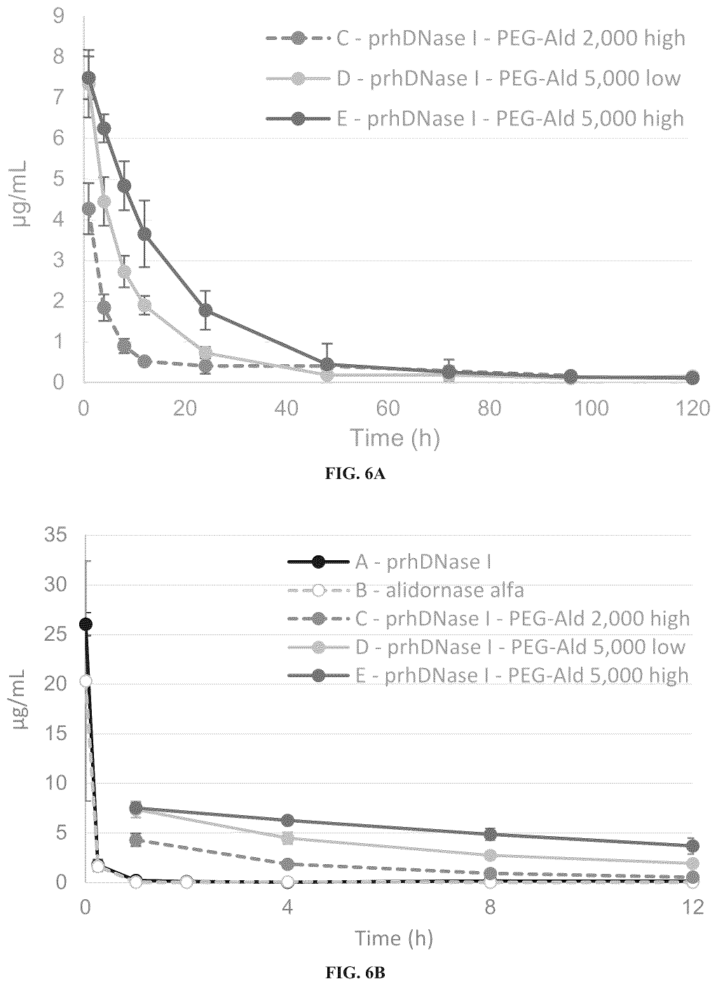

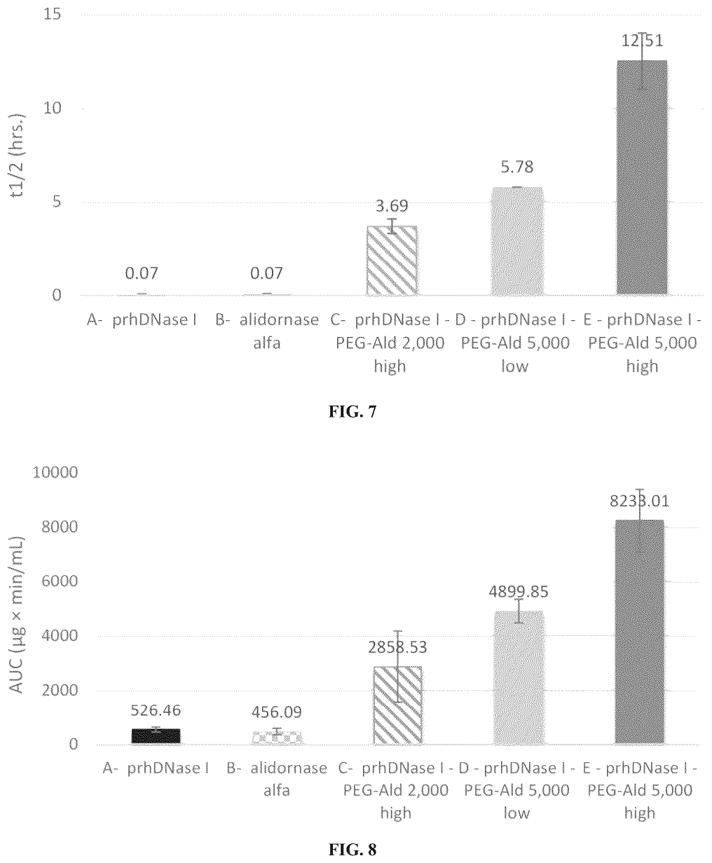

FIG. 4 shows that the half-life of an exemplary modified DNase protein in rats was about 10 hours (in contrast to about 7 minutes for the corresponding non-modified DNase protein). FIGS. 6A-7 show that the half-life of an exemplary modified DNase protein in rats was about 4 to 12.5 hours, depending on the degree of modification (in contrast to several minutes for corresponding non-modified DNase protein). FIG. 8 shows that the considerably increased half-life of the modified DNase protein is associated with a considerable increase in AUC (area under curve).

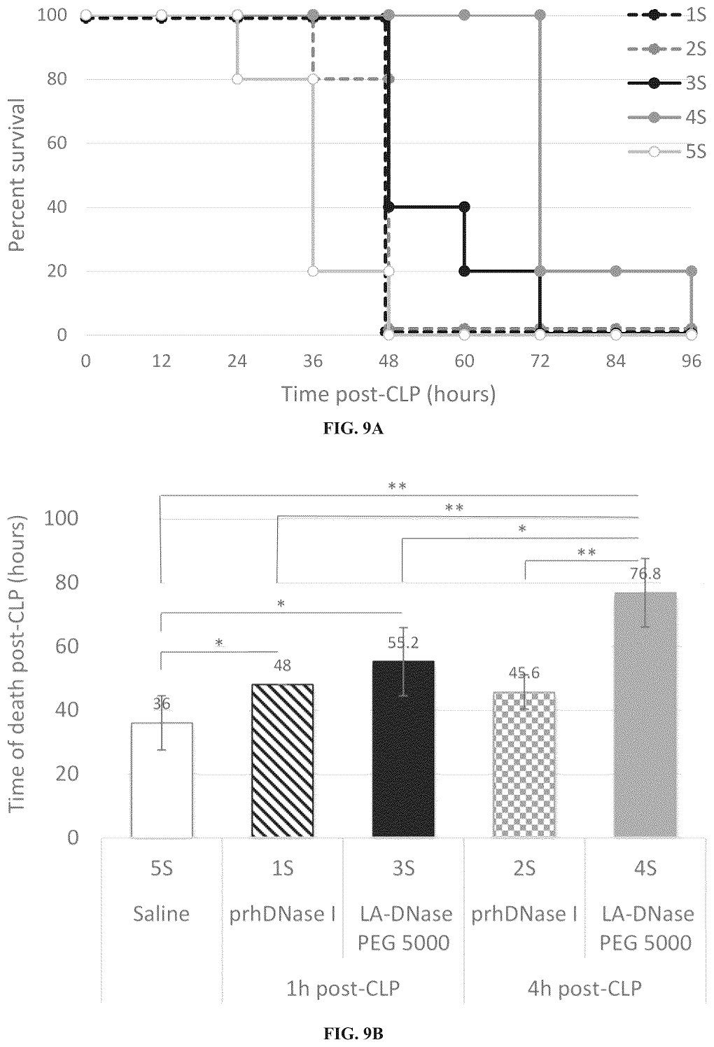

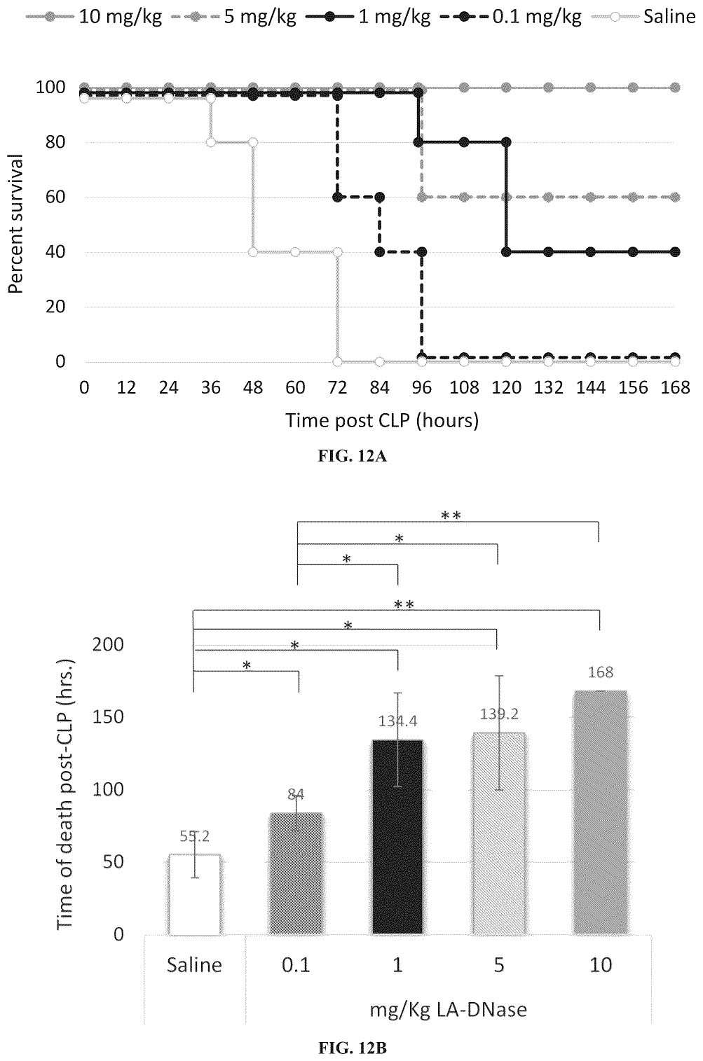

FIGS. 9A-12B show that an exemplary modified DNase protein exhibits a therapeutic effect in mice with sepsis. FIGS. 9A and 9B show that the modified DNase protein is more potent than the corresponding non-modified DNase protein. FIGS. 12A and 12B show that the therapeutic effect of the modified DNase protein is dose-dependent.

Modified DNase Protein:

According to an aspect of some embodiments of the invention, there is provided a modified DNase protein comprising a DNase polypeptide attached to at least two poly(alkylene glycol) moieties.

According to some of any of the embodiments of the invention, the polypeptide is attached to from 2 to 7 poly(alkylene glycol) moieties, optionally from 2 to 6 poly(alkylene glycol) moieties, optionally from 2 to 5 poly(alkylene glycol) moieties, optionally from 2 to 4 poly(alkylene glycol) moieties, and optionally from 2 to 3 poly(alkylene glycol) moieties.

In some of any of the embodiments of the invention, the polypeptide is attached to at least three poly(alkylene glycol) moieties, optionally from 3 to 7 poly(alkylene glycol) moieties, optionally from 3 to 6 poly(alkylene glycol) moieties, optionally from 3 to 5 poly(alkylene glycol) moieties, and optionally from 3 to 4 poly(alkylene glycol) moieties.

In some of any of the embodiments of the invention, the polypeptide is attached to at least four poly(alkylene glycol) moieties, optionally from 4 to 7 poly(alkylene glycol) moieties, optionally from 4 to 6 poly(alkylene glycol) moieties, and optionally from 4 to 5 poly(alkylene glycol) moieties.

In some of any of the respective embodiments, at least 10% of the amine groups comprised by lysine residue side chains and the N-terminus in the DNase polypeptide are attached to a poly(alkylene glycol) moiety (e.g., according to any of the embodiments described herein relating to a poly(alkylene glycol) moiety which attaches to a lysine residue side chain), and optionally at least 20%, at least 30%, at least 40%, at least 50%, at least 60%, at least 70%, at least 80%, at least 90%, and even about 100% of the lysine residue side chain and N-terminus gamine groups are attached to a poly(alkylene glycol) moiety.

Herein throughout, modified DNase protein encompasses populations of a modified DNase protein, and the number of poly(alkylene glycol) moieties attached to a polypeptide (according to any of the respective embodiments described herein) and/or the percentage of amine groups attached to a poly(alkylene glycol) moiety refers to an average (e.g., mean) number and/or percentage in the population.

In some of any of the embodiments described herein, the modified DNase protein is characterized by a longer in vivo half-life than a corresponding non-modified DNase protein (i.e., without the poly(alkylene glycol) moieties described herein). In some such embodiments described herein, the half-life of the modified DNase protein is at least 20% longer than that of the corresponding non-modified DNase protein. In some embodiments, the half-life of the modified DNase protein is at least 50% longer than that of the corresponding non-modified DNase protein. In some embodiments, the half-life of the modified DNase protein is at least 100% longer than—i.e., at least two-fold—that of the corresponding non-modified DNase protein. In some embodiments, the half-life of the modified DNase protein is at least three-fold that of the corresponding non-modified DNase protein. In some embodiments, the half-life of the modified DNase protein is at least five-fold that of the corresponding non-modified DNase protein. In some embodiments, the half-life of the modified DNase protein is at least 10-fold that of the corresponding non-modified DNase protein. In some embodiments, the half-life of the modified DNase protein is at least 20-fold that of the corresponding non-modified DNase protein. In some embodiments, the half-life of the modified DNase protein is at least 50-fold that of the corresponding non-modified DNase protein. In some embodiments, the half-life of the modified DNase protein is at least 100-fold that of the corresponding non-modified DNase protein. In some embodiments, the half-life of the modified DNase protein is at least 200-fold that of the corresponding non-modified DNase protein. In some embodiments, the half-life of the modified DNase protein is at least 500-fold that of the corresponding non-modified DNase protein.

A half-life of (modified and/or non-modified) DNase protein may be determined, for example, by determining an amount of the tested DNase protein in the blood (e.g., in plasma) over time, following injection of the tested DNase protein into a subject (e.g., in humans, in rats and/or in mice). As exemplified herein, an amount of DNase protein may be determined using an assay for DNA concentration (e.g., using salmon testis DNA) to evaluate a decrease in DNA concentration over time, for example, a spectrophotometric assay in which methyl green dye non-covalently attached to DNA changes color upon DNA hydrolysis (e.g., as described in the Examples section below). Alternatively or additionally, an amount of DNase protein may be determined using a suitable antibody, e.g., as an ELISA test.

In some of any of the embodiments described herein, the modified DNase protein is characterized by a plasma half-life (e.g., as determined by antibody recognition and/or enzymatic activity) in rats and/or mice of at least 1 hour. In some such embodiments, the half-life is at least 2 hours. In some embodiments, the half-life is at least 3 hours. In some embodiments, the half-life is at least 6 hours. In some embodiments, the half-life is at least 12 hours. In some embodiments, the half-life is at least 24 hours. In some embodiments, the half-life is at least two days, or at least three days, or at least one week.

A longer half-life of a modified DNase protein according to any of the respective embodiments described herein may optionally be associated with a greater molecular weight of the modified DNase protein (which may decrease a rate of removal from the bloodstream, e.g., by filtration in the kidneys) and/or by lower immunogenicity of the modified DNase protein (which may decrease a rate of inactivation and/or destruction by the immune system).

Poly(Alkylene Glycol) Moieties:

A poly(alkylene glycol) moiety according to any of the embodiments described herein may optionally be combined with a DNase polypeptide according to any of the embodiments described herein (e.g., in the respective section herein) in any manner described herein (e.g., according to any of the embodiments described herein relating to a nature of attachment of the poly(alkylene glycol) moieties to the DNase polypeptide.

The phrase “poly(alkylene glycol)”, as used herein, encompasses a family of polyether polymers which share the following general formula: —[O—(CH₂)m]n-O—, wherein m represents the number of methylene groups present in each alkylene glycol unit, and n represents the number of repeating units, and therefore represents the size or length of the polymer. For example, when m=2, the polymer is referred to as a polyethylene glycol, and when m=3, the polymer is referred to as a polypropylene glycol.

In some embodiments, m is an integer greater than 1 (e.g., m=2, 3, 4, etc.).

Optionally, m varies among the units of the poly(alkylene glycol) chain. For example, a poly(alkylene glycol) chain may comprise both ethylene glycol (m=2) and propylene glycol (m=3) units linked together.

The phrase “poly(alkylene glycol)” also encompasses analogs thereof, in which the oxygen atom is replaced by another heteroatom such as, for example, S, —NH— and the like. This term further encompasses derivatives of the above, in which one or more of the methylene groups composing the polymer are substituted. Examples of optional substituents on the methylene groups include, but are not limited to, alkyl, cycloalkyl, alkenyl, alkynyl, alkoxy, hydroxy, oxo, thiol and thioalkoxy, and the like. In some embodiments, substituents on the methylene groups (if any are present) are alkyl, optionally C₁₋₄-alkyl, and optionally methyl.

The phrase “alkylene glycol unit”, as used herein, encompasses a —O—(CH₂)m- group or an analog thereof, as described hereinabove, which forms the backbone chain of the poly(alkylene glycol), wherein the (CH₂)m (or analog thereof) is bound to an oxygen atom (or heteroatom analog thereof) at a terminus of a poly(alkylene glycol) (as indicated in the formula —[O—(CH₂)m]n-O—) or heteroatom analog thereof, or a heteroatom belonging to another alkylene glycol unit or to a DNase polypeptide (in cases of a terminal unit); and the 0 (or aforementioned terminal oxygen atom) or heteroatom analog thereof is bound to the (CH₂)m (or analog thereof) of another alkylene glycol unit, or to a functional group which forms a bond with a DNase polypeptide (according to any of the respective embodiments described herein).

An alkylene glycol unit may be branched, such that it is linked to 3 or more neighboring alkylene glycol units, wherein each of the 3 or more neighboring alkylene glycol units are part of a poly(alkylene glycol) chain. Such a branched alkylene glycol unit is linked via the heteroatom thereof to one neighboring alkylene glycol unit, and hetero atoms of the remaining neighboring alkylene glycol units are each linked to a carbon atom of the branched alkylene glycol unit. In addition, a heteroatom (e.g., nitrogen) may bind more than one carbon atom of an alkylene glycol unit of which it is part, thereby forming a branched alkylene glycol unit (e.g., [(—CH₂)m]₂N— and the like).

In exemplary embodiments, at least 50% of alkylene glycol units are identical, e.g., they comprise the same heteroatoms and the same m values as one another. Optionally, at least 70%, optionally at least 90%, and optionally 100% of the alkylene glycol units are identical. In exemplary embodiments, the heteroatoms bound to the identical alkylene glycol units are oxygen atoms and/or the alkylene glycol units are non-substituted. In further exemplary embodiments, m is 2 for the identical units.

In some of any of the respective embodiments, the poly(alkylene glycol) moiety comprises a polyethylene glycol (PEG) or analog thereof.

As used herein, the term “polyethylene glycol” describes a poly(alkylene glycol), as defined hereinabove, wherein at least 50%, at least 70%, at least 90%, and preferably 100%, of the alkylene glycol units are —CH₂CH₂—O—. Similarly, the phrase “ethylene glycol units” is defined herein as units of —CH₂CH₂O—.

In some of any of the respective embodiments, polyethylene glycol (PEG) or analog thereof has a general formula:

—(Y₁—CR₁R₂—CR₃R₄)n-Y₂—
wherein Y₁ and Y₂ are each independently O, S or NR₅ (optionally O);
n is an integer, optionally from 2 to 1000 (optionally from 10 to 300, and optionally from to 100), although higher values of n are also contemplated; and
each of R₁, R₂, R₃, R₄, and R₅ is independently hydrogen, alkyl, cycloalkyl, alkenyl, alkynyl, alkoxy, hydroxy, oxo, thiol and/or thioalkoxy.

In some of any of the respective embodiments, R₁, R₂, R₃, R₄, and R₅ each independently hydrogen or alkyl, optionally hydrogen or C₁₋₄-alkyl, and optionally hydrogen or methyl. In exemplary embodiments, R₁, R₂, R₃, R₄, and R₅ each hydrogen.

The polyethylene glycol or analog thereof may optionally comprise a copolymer, for example, wherein the Y₁—CR₁R₂—CR₃R₄ units in the above formula are not all identical to one another.

In some embodiments, at least 50% of Y₁—CR₁R₂—CR₃R₄ units are identical. Optionally, at least 70%, optionally at least 90%, and optionally 100% of the Y₁—CR₁R₂—CR₃R₄ units are identical.

Optionally, polyethylene glycol moiety is branched, for example, such that for one or more Y₁—CR₁R₂—CR₃R₄ units in the above formula, at least of one of R₁, R₂, R₃, R₄, and R₅ is —(Y₁—CR₁R₂—CR₃R₄)p-Y₂—, wherein R₁-R₅ and Y₁ and Y₂ are as defined hereinabove, and p is an integer as defined herein for n (e.g., from 2 to 1000) according to any of the respective embodiments.

Each poly(alkylene glycol) moiety may optionally comprise a functional group forming a covalent bond with a DNase polypeptide. Examples of functional groups include an alkylene group and a carbonyl (—C(═O)—). The alkylene or carbonyl may optionally be attached to a nitrogen atom (e.g., of an amine group) of the polypeptide, e.g., so as to together form an amine group or amide group, respectively). Each functional group may optionally be attached directly to a poly(alkylene glycol) moiety (according to any of the respective embodiments described herein, or indirectly via a linking group, optionally wherein the linking group is a hydrocarbon moiety.

Herein throughout, the phrase “linking group” describes a group (e.g., a substituent) that is attached to two or more moieties in the compound; whereas the phrase “end group” describes a group (e.g., a substituent) that is attached to a single moiety in the compound via one atom thereof.

Each poly(alkylene glycol) moiety may independently be covalently attached to the DNase polypeptide at one or more site.

In some of any of the embodiments of the invention, at least a portion, or each, of the poly(alkylene glycol) moieties are monofunctional poly(alkylene glycol) moieties. A “monofunctional” moiety refers to a moiety that is covalently attached to one site (and no more). Thus, a linear monofunctional moiety is terminated by an end group, as this term is defined herein (for example, hydrogen or a hydrocarbon moiety, optionally methyl) at a terminus distal to the covalent attachment; whereas a branched monofunctional moiety comprises two or more termini with such an end group (wherein the functional groups at the different termini may be the same or different).

In some of any of the embodiments of the invention, at least a portion, or each, of the poly(alkylene glycol) moieties have formula I:

-L₂-L₁-[O—(CH₂)m]n-O—R₁ Formula I
wherein:
L₁ and L₂ are each independently a hydrocarbon moiety or absent;
R₁ is hydrogen or a hydrocarbon moiety;
m is an integer of at least 2, optionally in a range of from 2 to 10; and
n is an integer of at least 2, optionally in a range of from 2 to 1000.

In some of any of the respective embodiments described herein, L₁ and L₂ are each independently a substituted or non-substituted alkylene, optionally being from 1 to 6 carbon atoms in length, optionally from 1 to 4 carbon atoms in length, optionally from 1 to 3 carbon atoms in length, and optionally 1 or 2 carbon atoms in length. In some such embodiments, the alkylene is non-substituted, for example, CH₂ or CH₂CH₂.

In some of any of the respective embodiments of the invention, for at least a portion, or each, of the poly(alkylene glycol) moieties, L₂ is CH₂, such that moiety has formula I′:

—CH₂-L₁-[O—(CH₂)m]n-O—R₁ Formula I′

wherein L₁, R₁, m and n are defined as for Formula I (according to any of the respective embodiments described herein).

In some of any of the embodiments herein relating to a formula including a variable m, is 2, 3 or 4. In some embodiments, m is 2 or 3. In some embodiments, m is 2, such that the poly(alkylene glycol) moiety comprises a polyethylene glycol moiety (with n ethylene glycol subunits).

In some of any of the embodiments herein relating to a formula including a variable n, n is at least 10 (e.g., from 10 to 300, or from 10 to 200, or from 10 to 150, or from 10 to 100, or from or from 10 to 80, or from or from 10 to 60). In some such embodiments, n is at least 20 (e.g., from 20 to 300, or from 20 to 200, or from 20 to 150, or from or from 20 to 100, or from or from 20 to 80, or from or from 20 to 60). In some embodiments, n is at least 30 (e.g., from 30 to 300, or from 30 to 200, or from 30 to 150, or from or from 30 to 100, or from or from 30 to 80, or from or from 30 to 60). In some embodiments, n is at least 40 (e.g., from 40 to 300, or from 40 to 200, or from 40 to 150, or from or from 40 to 100, or from or from 40 to 80, or from or from 40 to 60). In some embodiments, n is at least 50 (e.g., from 50 to 300, or from 50 to 200, or from 50 to 150, or from or from 50 to 100, or from or from 50 to 80). In some embodiments, n is at least 60 (e.g., from 60 to 300, or from 60 to 200, or from 60 to 150, or from or from 60 to 100, or from or from 60 to 80). In some embodiments, n is at least 70 (e.g., from 70 to 300, or from 70 to 200, or from 70 to 150, or from or from 70 to 100).

In some of any of the embodiments herein relating to a formula including variables m and n, n is at least 10 (e.g., from 10 to 300, or from 10 to 200, or from 10 to 150, or from 10 to 100, or from or from 10 to 80, or from or from 10 to 60); and m is 2, 3 or 4, preferably 2 or 3, and more preferably 2. In some such embodiments, n is at least 20 (e.g., from 20 to 300, or from 20 to 200, or from 20 to 150, or from or from 20 to 100, or from or from 20 to 80, or from or from 20 to 60). In some embodiments, n is at least 30 (e.g., from 30 to 300, or from 30 to 200, or from 30 to 150, or from or from 30 to 100, or from or from 30 to 80, or from or from 30 to 60). In some embodiments, n is at least 40 (e.g., from 40 to 300, or from 40 to 200, or from 40 to 150, or from or from 40 to 100, or from or from 40 to 80, or from or from 40 to 60). In some embodiments, n is at least 50 (e.g., from 50 to 300, or from 50 to 200, or from 50 to 150, or from or from 50 to 100, or from or from 50 to 80). In some embodiments, n is at least 60 (e.g., from 60 to 300, or from 60 to 200, or from 60 to 150, or from or from 60 to 100, or from or from 60 to 80). In some embodiments, n is at least 70 (e.g., from 70 to 300, or from 70 to 200, or from 70 to 150, or from or from 70 to 100).

In some of any of the embodiments of the invention, at least a portion, or each, of the poly(alkylene glycol) (optionally monofunctional poly(alkylene glycol)) moieties comprise an alkylene group (e.g., a non-substituted alkylene group) covalently attached to a nitrogen atom of an amine group in the polypeptide; for example, an amine group of a lysine residue side chain and/or an N-terminus. The alkylene (attached to a nitrogen atom) may optionally be, for example, L₂ according to formula I, L₁ (wherein L₂ is absent) according to formula I, and/or a terminal CH₂ group according to formula I′ (optionally in combination with at least a portion of L₁), according to any of the respective embodiments described herein.

As exemplified herein, such an alkylene group covalently attached to a nitrogen atom may optionally be obtained by reacting an aldehyde group with an amine group in the presence of a reducing agent (e.g., according to a process described herein).

Without being bound by any particular theory, it is believed that attachment of a poly(alkylene glycol) moiety via an alkylene group covalently attached to a polypeptide nitrogen atom is advantageously less immunogenic and/or less deleterious to enzymatic activity than alternative techniques for covalent attachment, such as forming an amide bond between a carbonyl (—C(═O)—) group (optionally derived by condensation of a carboxylate group) and a polypeptide amine group.

In some of any of the respective embodiments described herein, a molecular weight of the poly(alkylene glycol) moiety (optionally a monofunctional poly(alkylene glycol) moiety) is no more than about 10 kDa. In some such embodiments, the molecular weight of the poly(alkylene glycol) moiety is no more than about 7.5 kDa. In some embodiments, the molecular weight of the poly(alkylene glycol) moiety is no more than about 5 kDa.

In some of any of the respective embodiments described herein, a molecular weight of the poly(alkylene glycol) moiety (optionally a monofunctional poly(alkylene glycol) moiety) is at least about 1.5 kDa. In some such embodiments, the molecular weight of the poly(alkylene glycol) moiety is in a range of from about 1.5 kDa to about 10 kDa. In some embodiments, the molecular weight of the poly(alkylene glycol) moiety is in a range of from about 1.5 kDa to about 7.5 kDa. In some embodiments, the molecular weight of the poly(alkylene glycol) moiety is in a range of from about 1.5 kDa to about 5 kDa. In some embodiments, the molecular weight of the poly(alkylene glycol) moiety is in a range of from about 1.5 kDa to about 3 kDa.

In some of any of the respective embodiments described herein, a molecular weight of the poly(alkylene glycol) moiety (optionally a monofunctional poly(alkylene glycol) moiety) is at least about 2 kDa. In some such embodiments, the molecular weight of the poly(alkylene glycol) moiety is in a range of from about 2 kDa to about 10 kDa. In some embodiments, the molecular weight of the poly(alkylene glycol) moiety is in a range of from about 2 kDa to about 7.5 kDa. In some embodiments, the molecular weight of the poly(alkylene glycol) moiety is in a range of from about 2 kDa to about 5 kDa. In some embodiments, the molecular weight of the poly(alkylene glycol) moiety is in a range of from about 2 kDa to about 3 kDa. In some exemplary embodiments, the molecular weight of the poly(alkylene glycol) moiety is about 2 kDa.

In some of any of the respective embodiments described herein, a molecular weight of the poly(alkylene glycol) moiety (optionally a monofunctional poly(alkylene glycol) moiety) is at least about 3 kDa. In some such embodiments, the molecular weight of the poly(alkylene glycol) moiety is in a range of from about 3 kDa to about 10 kDa. In some embodiments, the molecular weight of the poly(alkylene glycol) moiety is in a range of from about 3 kDa to about 7.5 kDa. In some embodiments, the molecular weight of the poly(alkylene glycol) moiety is in a range of from about 3 kDa to about 5 kDa. In some embodiments, the molecular weight of the poly(alkylene glycol) moiety is in a range of from about 2 kDa to about 3 kDa.

In some of any of the respective embodiments described herein, a molecular weight of the poly(alkylene glycol) moiety (optionally a monofunctional poly(alkylene glycol) moiety) is at least about 4 kDa. In some such embodiments, the molecular weight of the poly(alkylene glycol) moiety is in a range of from about 4 kDa to about 10 kDa. In some embodiments, the molecular weight of the poly(alkylene glycol) moiety is in a range of from about 4 kDa to about 7.5 kDa. In some embodiments, the molecular weight of the poly(alkylene glycol) moiety is in a range of from about 4 kDa to about 5 kDa.

In some of any of the respective embodiments described herein, a molecular weight of the poly(alkylene glycol) moiety (optionally a monofunctional poly(alkylene glycol) moiety) is at least about 5 kDa. In some such embodiments, the molecular weight of the poly(alkylene glycol) moiety is in a range of from about 5 kDa to about 10 kDa. In some embodiments, the molecular weight of the poly(alkylene glycol) moiety is in a range of from about 5 kDa to about 7.5 kDa. In some embodiments, the molecular weight of the poly(alkylene glycol) moiety is about 5 kDa.

Without being bound by any particular theory, it is believed that poly(alkylene glycol) moiety can mask the DNase polypeptide from the immune system in a manner which protects the DNase activity in vivo and/or decreases immunogenicity, and that an excessively small poly(alkylene glycol) moiety and/or small number (e.g., one) of poly(alkylene glycol) moieties of may result in ineffective masking of the polypeptide. It is further believed that an excessively large poly(alkylene glycol) moiety may result in ineffective masking of the polypeptide, for example, wherein attachment of a large poly(alkylene glycol) moiety sterically inhibits attachment of an additional poly(alkylene glycol) moiety, leaving gaps in the masking of the polypeptide (e.g., through which antibodies may penetrate). It is further believed that a large poly(alkylene glycol) moiety may itself be more immunoreactive than shorter moieties [Rudmann et al., Toxicologic Pathology 2013, 41:970-983; Moreno et al., Cell Chem Biol 2019, 26:634-644; Garay et al., Expert Opin Drug Deliv 2012, 9:1319-1323; Wan et al., Process Biochemistry 2017, 52:183-191; Ehrlich et al., J Mol Recognition 2009, 22:99-103].

Decreasing immunogenicity may facilitate the repeated administration of the modified protein and/or enhance the efficacy of the protein. For example, anti-PEG antibodies were reported as a reason for PEGylated uricase losing activity during clinical treatment [Zhang et al., J Control Release 2016, 244:184-193]. Moreover, even acute treatment options may be jeopardized by the presence of pre-existing antibodies against poly(alkylene glycol) moieties reported to be present in the general population [Lubich et al., Pharm Res 2016, 33:2239-2249].

In addition, use of relatively short poly(alkylene glycol) moieties may optionally allow better control over the circulating time of a modified DNase protein, by allowing more flexibility in determining a degree of modification (e.g., by modulating number of poly(alkylene glycol) moieties and/or poly(alkylene glycol) moiety size, as exemplified herein), thereby facilitating tailoring a long-acting modified DNase to the specific needs of treatment of different indications. For example, a relatively short half-life (e.g., about a day or less) may be most suitable for treating an acute condition (e.g., an acute condition associated with an inflammation); whereas a longer half-life may be most suitable for treating a chronic condition (e.g., to allow for less frequent administration).

DNase Polypeptide:

Except where modified DNase protein is explicitly referred to, the following section described a DNase polypeptide which corresponds to the modified DNase protein described herein except for the presence of the poly(alkylene glycol) moieties described herein, according to any one of the embodiments.

In the context of a non-modified protein, the terms “protein” and “polypeptide are used herein interchangeably”. In the context of a modified protein, the term “polypeptide” is merely used to emphasize the portion of the modified protein derived from the non-modified protein, as opposed to the poly(alkylene glycol) moieties, and is not intended to be limiting.

The skilled person will understand the structure of a modified DNase protein according to some embodiments of the invention by considering the non-modified DNase according to any one of the embodiments described in this section in combination with a modification (e.g., PEGylation) thereof according to any one of the respective embodiments described herein.

As used herein the terms “DNase” and “DNase protein” encompass any deoxyribonuclease, including DNase I and DNase II families of deoxyribonuclease.

As used herein the terms “DNase I” and “DNase I protein” refer to a deoxyribonuclease I (EC 3.1.21.1) polypeptide. DNase I is classified as an endonuclease, which cleaves DNA to produce 5′-phosphodinucleotide and 5′-phosphooligonucleotide end products, with a preference for double stranded DNA substrates and alkaline pH optimum.

DNase I acts on single-stranded DNA, double-stranded DNA, and chromatin.

As used herein the terms “DNase II” and “DNase II protein” refer to a deoxyribonuclease II (EC 3.1.22.1) polypeptide. DNase II is classified as an endonuclease, with a preference for acid pH optimum.

The DNase according to some embodiment of the present teachings (i.e., non-modified) is inhibited by actin.

The DNase according to some embodiment of the present teachings (i.e., non-modified) is not inhibited by actin.

Herein, the phrase “inhibited by actin” refers to a reduction of at least 20% in a DNA hydrolytic activity (e.g., of a DNase enzyme) in the presence of 50 μg/mL human non-muscle actin (relative to the activity in the absence of actin) at 37° C.

In some of any of the respective embodiments described herein, the DNase is a DNase I, as defined herein.

According to a specific embodiment, the DNase is human DNase I as set forth in SEQ ID NO: 1.

Also contemplated are homologs (i.e., functional equivalents) and orthologs (e.g., mouse NM_010061.5 NO_034191.3) of the human DNase I having the DNase I activity.

Herein, a “homolog” of a given polypeptide refers to a polypeptide that exhibits at least % homology, preferably at least 90% homology, and more preferably at least 95% homology, and more preferably at least 98% homology to the given polypeptide (optionally exhibiting at least 80%, at least 90% identity, at least 95%, or at least 98% sequence identity to the given polypeptide). In some embodiments, a homolog of a given polypeptide further shares a therapeutic activity with the given polypeptide. The percentage of homology refers to the percentage of amino acid residues in a first polypeptide sequence which matches a corresponding residue of a second polypeptide sequence to which the first polypeptide is being compared. Generally, the polypeptides are aligned to give maximum homology. A variety of strategies are known in the art for performing comparisons of amino acid or nucleotide sequences in order to assess degrees of identity, including, for example, manual alignment, computer assisted sequence alignment and combinations thereof. A number of algorithms (which are generally computer implemented) for performing sequence alignment are widely available, or can be produced by one of skill in the art. Representative algorithms include, e.g., the local homology algorithm of Smith and Waterman [Adv Appl Math, 1981, 2:482]; the homology alignment algorithm of Needleman and Wunsch [J Mol Biol 1970, 48:443]; the search for similarity method of Pearson and Lipman [PNAS 988, 85:2444]; and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.). Readily available computer programs incorporating such algorithms include, for example, BLASTN, BLASTP, Gapped BLAST, PILEUP, CLUSTALW etc. When utilizing BLAST and Gapped BLAST programs, default parameters of the respective programs may be used. Alternatively, the practitioner may use non-default parameters depending on his or her experimental and/or other requirements (see for example, the Web site having URL www(dot)ncbi(dot)nlm(dot)nih(dot)gov).

Such homologs can be, for example, at least 80%, at least 81%, at least 82%, at least 83%, at least 84%, at least 85%, at least 86%, at least 87%, at least 88%, at least 89%, at least %, at least 91%, at least 92%, at least 93%, at least 94%, at least 95%, at least 96%, at least 97%, at least 98%, at least 99% or 100% identical to SEQ ID NO: 1 or homologous (identity+homology), as determined using the BestFit software of the Wisconsin sequence analysis package, utilizing the Smith and Waterman algorithm, where gap weight equals 50, length weight equals 3, average match equals 10 and average mismatch equals −9.

Embodiments of the invention encompass nucleic acid sequences described hereinabove; fragments thereof, sequences hybridizable therewith, sequences homologous thereto, sequences orthologous thereto, sequences encoding similar polypeptides with different codon usage, altered sequences characterized by mutations, such as deletion, insertion or substitution of one or more nucleotides, either naturally occurring or man-induced, either randomly or in a targeted fashion, all of which are collectively termed “substantial homologs”).

The term “substantial homolog”, when used to describe the amino acid sequence of a DNase protein which is modified to provide the modified DNase, also refers herein to an amino acid sequence having at least 80% homology, optionally at least 90% homology, optionally at least 95% homology, optionally at least 98% homology, and optionally at least 99% homology to another amino acid sequence of a DNase protein as described in detail herein.

Other members of the DNase I family of endonucleases are DNase X, DNase gamma, DNase lambda, DNase1L2, DNase1L3 and tear lipocalin in humans. DNase I also encompasses, inter alia, alkaline DNase, bovine pancreatic (bp) DNase, DNase A, DNA phosphatase and DNA endonuclease, for example, in Bos taurus.

The non-modified DNase can be a purified DNase which is extracted from a cell/tissue in which it is naturally expressed.

Alternatively or additionally, the DNase is recombinantly produced. In some embodiments, the DNase is a recombinantly produced DNase I. In some embodiments, the DNase (e.g., DNase I) is a plant recombinant polypeptide, that is, recombinantly produced by a plant cell.

For recombinant expression, the nucleic acid sequence encoding DNase is ligated into a nucleic acid expression vector under the transcriptional regulation of a cis-acting regulatory element e.g., a promoter.

Other than containing the necessary elements for the transcription and translation of the inserted coding sequence, the expression construct of some embodiments of the invention can also include sequences engineered to enhance stability, production, purification, yield or toxicity of the expressed peptide. A variety of prokaryotic or eukaryotic cells can be used as host-expression systems to express the DNase of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the polypeptides of some embodiments of the invention.

Examples of bacterial constructs include the pET series of E. coli expression vectors [Studier et al., Methods Enzymol 1990, 185:60-89].

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 19S RNA promoters of CaMV [Brisson et al., Nature 1984, 310:511-514], or the coat protein promoter to TMV [Takamatsu et al., EMBO J 1987, 6:307-311] can be used. Alternatively, plant promoters such as the small subunit of RUBISCO [Coruzzi et al., EMBO J 1984, 3:1671-1680; Brogli et al., Science 1984, 224:838-843] or heat shock promoters, e.g., soybean hsp17.5-E or hsp17.3-B [Gurley et al., Mol Cell Biol 1986, 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, Methods for Plant Molecular Biology (1988), Academic Press, NY, Section VIII, pp 421-463.

According to a specific embodiment, the DNase is produced in a plant cell suspension culture as described in International Patent Application Publication WO 2013/114374, which is hereby incorporated by reference in its entirety.

Accordingly, at least a portion of the human DNase I protein has an N-terminal glycine residue (SEQ ID NO: 2). In some embodiments, the human DNase I protein comprises a mixture of DNase I as set forth in SEQ ID NO: 2 and DNase I as set forth in SEQ ID NO: 1. Such a protein may be expressed from a nucleic acid construct which comprises a nucleic acid sequence encoding human DNase I translationally fused at the N-terminus thereof to an Arabidopsis ABPI endoplasmic reticulum targeting signal peptide encoded by a nucleic acid sequence as set forth in SEQ ID NO: 3.

As used herein, the term “Arabidopsis ABPI endoplasmic reticulum targeting signal peptide” refers to the leader peptide sequence of the Arabidopsis thaliana auxin binding protein, which is capable of directing the expressed protein to the endoplasmic reticulum within the plant cell. In one embodiment, the Arabidopsis ABPI endoplasmic reticulum targeting signal peptide is a 33 amino acid polypeptide as set forth in SEQ ID NO: 8.

Thus, according to some embodiments, the human DNase I protein contiguously linked at the N-terminal to an Arabidopsis ABPI endoplasmic reticulum targeting signal peptide and the human DNase I protein has an amino acid sequence as set forth in SEQ ID NO: 9.

The human DNase I protein may optionally be encoded by a nucleic acid sequence as set forth in SEQ ID NO: 6. The Arabidopsis ABPI endoplasmic reticulum targeting signal peptide may optionally be encoded by a nucleic acid sequence as set forth in SEQ ID NO: 3. A human DNase I protein contiguously linked at the N-terminal to an Arabidopsis ABPI endoplasmic reticulum targeting signal peptide may optionally be encoded by a nucleic acid sequence as set forth in SEQ ID NO: 7.

Further presented herein are a native nucleic acid sequence (SEQ ID NO: 4) encoding a native human DNase I protein (SEQ ID NO: 5; GenBank: NM 005223, sequence (a)) which includes the native signal leader peptide.

Other expression systems such as insects and mammalian host cell systems which are well known in the art and are further described herein below can also be used by some embodiments of the invention.

According to some embodiments of any of the embodiments described herein relating to a human DNase I, the DNase I is mature human DNase I. In some embodiments, the DNase I is dornase alfa DNase I (e.g., Pulmozyme®).

According to some embodiments of any of the embodiments described herein, the human DNase I comprises an amino acid sequence as set forth in SEQ ID NO: 1.

It will be appreciated that a DNase I protein having an amino acid sequence homologous (e.g., at least 80% homologous, as described herein) to the human DNase I amino acid sequence of SEQ ID NO: 1 may optionally maintain characteristic structure and/or function of the human DNase I. One non-limiting example of an amino acid sequence homologous to an amino acid sequence of a human DNase I protein is SEQ ID NO: 2, which is closely similar to SEQ ID NO: 1.

In some embodiments of any of the embodiments described herein, the DNase protein is a variant human DNase I protein, optionally a naturally occurring (in at least some humans) variant of human DNase I. Variant human DNase proteins, having altered catalytic and/or other biochemical and structural properties, such as altered actin affinity, cofactor requirements, pH optimum, increased shelf life in storage and the like, enhanced recombinant expression or fusion proteins have been disclosed. Suitable modified DNase polypeptides include, but are not limited to DNase polypeptides disclosed in U.S. Pat. Nos. 6,348,343, 6,391,607, 7,407,785 and 7,297,526, and in International Patent Application Publications WO 96/26279, WO 2008/039989 and WO 2013/114374, each of which is incorporated by reference in its entirely, especially with respect to DNase polypeptides and methods of preparing them.

In some embodiments, the DNase is expressed in tobacco (e.g., Nicotiana tabacum cells), which may optionally be in suspension, for example, DNase I expressed in Bright Yellow-2 (BY2) cell culture (e.g., as exemplified herein below, and/or as described in International Patent Application Publication WO 2013/114374).

In some embodiments, Agrobacterium-mediated transformation is used to introduce foreign genes into a plant cell genome. This technique is based on the natural capability of the Agrobacterium to transform plant cells by transferring a plasmid DNA segment, the transferred DNA (T-DNA), into the host cell genome. Using this approach, a T-DNA molecule, consisting of a foreign gene and its regulatory elements, is randomly introduced into the plant genome. The site of integration, as well as the copy number of the gene insertions is not controlled, thus the transformation process results in a “pool” of transgenic cells composed of cells with various levels of expression of the transgene. The transgenic “pool” is subsequently used for clone isolation. Clone isolation results in the establishment of many single cell lines, from which the clone with the highest expression level of the foreign gene is then selected. In some embodiments the Agrobacterium-mediated transformation is used to introduce foreign genes into a genome of a tobacco cell, such as, but not limited to Nicotiana tabacum L. cv Bright Yellow (BY-2) cells.

In some embodiments of any of the embodiments described herein, molecular mass of the DNase (e.g., plant-recombinant human DNase I) polypeptide is similar to the molecular mass, as measured by PAGE and/or mass spectrometry, of recombinant human DNase I expressed in mammalian cells (Pulmozyme® DNase I).

In some embodiments of any of the embodiments described herein, the DNase (e.g., plant-recombinant human DNase I) polypeptide has a molecular mass of about 30 kDa, as measured by SDS-PAGE, and about 32 kDa, as measured by mass spectrometry.

In some embodiments of any of the embodiments described herein, the non-modified DNase (e.g., plant-recombinant human DNase I) is glycosylated.

In some embodiments of any of the embodiments described herein, the modified DNase (e.g., plant-recombinant human DNase I) is glycosylated.

In some embodiments of any of the embodiments described herein, the isoelectric point of the glycosylated DNase (e.g., plant-recombinant human DNase I) protein is at a higher pH than that of recombinant human DNase I expressed in mammalian cells (Pulmozyme®).

When a range of isoelectric points occurs (e.g., a band is observed upon isoelectric focusing), the “isoelectric point” of a DNase refers herein to an average isoelectric point.

Without being bound by any particular theory, it is believed that a higher isoelectric point (suggesting a less negative charge) in comparison to DNase expressed in mammalian cells (as exemplified herein with plant recombinant DNase I) and/or retention of positively charged amine groups (e.g., as in reductive amination versus amide bond formation), may enhance affinity of the DNase to negatively charged DNA, thereby reducing the Michaelis constant.

In some embodiments of any of the embodiments described herein, the DNase (e.g., plant-recombinant human DNase I) is a glycosylated protein, comprising a polypeptide moiety having a molecular mass of about 29 kDa.

In some embodiments of any of the embodiments described herein, the modified and/or non-modified DNase is a purified protein, optionally characterized by a purity (e.g., of DNase I in a composition described herein) of at least 85%, at least 87%, at least 90%, at least 91%, at least 91.5%, at least 92%, at least 92.5%, at least 93%, at least 93.1%, at least 93.2%, at least 93.3%, at least 93.4%, at least 93.5%, at least 93.6%, at least 93.7%, at least 93.8%, at least 93.9%, at least 94%, at least 94.5%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, in a range of at least 95.0-99.8% or 100% purity. In some embodiments, purity of the modified and/or non-modified DNase protein is measured by HPLC.

The purity described hereinabove refers to low levels (or absence) of impurities. Ingredients deliberately added to a composition comprising modified and/or non-modified DNase (e.g., any ingredients of a composition such as described herein) are not considered herein as impurities which affect the purity of the DNase protein.

In some embodiments, the DNase is a recombinant DNase, optionally a plant-recombinant human DNase, and the purity described hereinabove refers to low levels (or absence) of impurities derived from the medium into which the DNase protein is secreted and/or from the host cell (e.g., plant host cell), such as, but not limited to nucleic acids and polynucleotides, amino acids, oligopeptides and polypeptides, glycans and other carbohydrates, lipids and the like. In some embodiments the host-cell derived impurities comprise biologically active molecules, such as enzymes.

In some embodiments of any one of the embodiments described herein, the DNase protein (e.g., plant-recombinant DNase, for example, plant-recombinant DNase I) is glycosylated, such that a DNase polypeptide has an average of at least 0.2, optionally at least 0.5, optionally at least one, optionally at least two, optionally at least three or optionally at least four or more exposed mannose residues per polypeptide molecule.

Herein, an “exposed” residue refers to a monosaccharide residue attached to a non-reducing end of a glycan by only one covalent bond.

In some embodiments of any one of the embodiments described herein, the DNase protein (e.g., plant-recombinant DNase, for example, plant-recombinant DNase I) is glycosylated, such that a DNase polypeptide has an average of at least one, and optionally at least two, core xylose residues per polypeptide molecule.

In some embodiments of any one of the embodiments described herein, the DNase protein (e.g., plant-recombinant DNase, for example, plant-recombinant DNase I) is glycosylated, such that a DNase polypeptide has an average of at least 0.2, optionally at least 0.5, optionally at least one, and optionally about two, core α-(1,3) fucose residues per polypeptide molecule.

In some embodiments of any one of the embodiments described herein, the DNase protein (e.g., plant-recombinant DNase, for example, plant-recombinant DNase I) is glycosylated, such that a DNase polypeptide has an average of at least one core xylose residue and at least one α-(1,3) fucose residue per polypeptide molecule.

In some embodiments of any one of the embodiments described herein, the DNase protein (e.g., plant-recombinant DNase, for example, plant-recombinant DNase I) is glycosylated, such that a DNase polypeptide has an average of at least one exposed mannose residue, at least one core xylose residue and at least one α-(1,3) fucose residue per polypeptide molecule.

In some embodiments of any one of the embodiments described herein, the DNase protein (e.g., plant-recombinant DNase, for example, plant-recombinant DNase I) is glycosylated, such that a DNase polypeptide has an average of at least one, optionally at least two, optionally at least 3, and optionally at least 4 terminal N-acetyl glucosamine substitutions per polypeptide molecule, optionally on the outer portion (distal from the polypeptide) of mannose residues.

In some embodiments of any one of the embodiments described herein, the DNase protein (e.g., plant-recombinant DNase, for example, plant-recombinant DNase I) is devoid of sialic acid residues.

Herein, “devoid of sialic acid residues” means that less than 1% of glycans contain a sialic acid residue, optionally less than 0.1%, and optionally less than 0.01%.

Some or all of the abovementioned characteristics regarding glycosylation may be obtained in plant-recombinant DNase (according to any of the respective embodiments described herein), which may optionally exhibit high mannose glycosylation (e.g., exposed mannose sugar residues and/or more than 3 mannose residues per glycan) and plant specific glycan residues.

Additional modifications (other than attachment of poly(alkylene glycol) moieties) may optionally be introduced to the DNase according to any of the embodiments described herein, optionally a modification which enhances actin resistance. Non-limiting examples include modifications (e.g., replacement of a carboxylic acid moiety with an amide moiety) such as described in International Patent Application Publication WO 2016/108244, the contents of which are incorporated herein in their entirety, particularly contents regarding modifications, and more particularly, modifications for enhancing actin resistance.

Preparation of Modified DNase:

According to an aspect of some embodiments of the invention, there is provided a process of preparing a modified DNase protein according to some of any of the respective embodiments described herein. The process, according to these embodiments, comprises: (a) contacting the DNase polypeptide (e.g., a DNase protein according to an of the embodiments described herein) with an agent that comprises a poly(alkylene glycol) (e.g., according to any of the respective embodiments described herein) attached to an aldehyde (—C(═O)H) group, to obtain a conjugate of the polypeptide and the agent comprising a poly(alkylene glycol); and (b) contacting the conjugate with a reducing agent.

In some of any of the respective embodiments described herein, the poly(alkylene glycol) comprises no more than one aldehyde group.

According to some of any of the embodiments of the invention relating to a process, the agent that comprises a poly(alkylene glycol) has formula II:

HC(═O)-L₁-[O—(CH₂)m]n-O—R₁ Formula II

wherein L₁ is a hydrocarbon moiety; R₁ is hydrogen or a hydrocarbon moiety; m is an integer in a range of at least 2, optionally from 2 to 10; and n is an integer of at least 2, optionally in a range of from 2 to 1000 (e.g., wherein L₁, R₁, m and/or n are as defined according to any of the respective embodiments described herein relating to formula I and/or I′). An agent of formula II may optionally be used to obtain a poly(alkylene glycol) moiety according to formula I and/or I′ (according to any of the respective embodiments described herein); for example, upon reaction of the aldehyde group with an amine group (e.g., to form an imine or hemiaminal intermediate), and reduction to form an amine group.

Examples of suitable reducing agents include, without limitation, borane and complexes thereof (e.g., picoline borane complex), borohydrides (e.g., sodium borohydride), triacetoxyborohydrides (e.g., sodium triacetoxyborohydride) cyanoborohydrides (e.g., sodium cyanoborohydride), and any other reducing agent known in the art to be suitable for a reductive amination process. Exemplary reducing agents include, without limitation, a 2-picoline borane complex, and sodium cyanoborohydride.

In some of any of the embodiments of the invention relating to a process, contacting the conjugate with a reducing agent is effected at a pH of at least about 7, and optionally at least about 8. In exemplary embodiments, the pH is about 7.

Without being bound by any particular theory, it is believed that higher pH values are generally associated with more DNase amine groups (e.g., of lysine residues) being active, and thus with more poly(alkylene glycol) moieties being attached to DNase polypeptide.

The DNase polypeptide, agent comprising a poly(alkylene glycol), and reducing agent may optionally be combined in any order. For example, an agent comprising a poly(alkylene glycol) may optionally be added to a mixture comprising the polypeptide and reducing agent, or the polypeptide may optionally be added to a mixture comprising the agent comprising a poly(alkylene glycol) and reducing agent (e.g., such that a conjugate of the polypeptide and agent comprising a poly(alkylene glycol) is already in contact with the reducing agent upon formation of the conjugate). In some embodiments, the DNase polypeptide, agent comprising a poly(alkylene glycol), and reducing agent are combined essentially concomitantly (e.g., as a “one-pot reaction”).

In some of any of the respective embodiments described herein, a molar ratio of the agent (according to any of the respective embodiments described herein) to the DNase polypeptide contacted with the agent (according to any of the respective embodiments described herein) is at least 10:1. In some such embodiments, the molar ratio is from 10:1 to 10,000:1. In some embodiments, the molar ratio is from 10:1 to 5,000:1. In some embodiments, the molar ratio is from 10:1 to 2,000:1. In some embodiments, the molar ratio is from 10:1 to 1,000:1. In some embodiments, the molar ratio is from 10:1 to 500:1. In some embodiments, the molar ratio is from 10:1 to 200:1. In some embodiments, the molar ratio is from 10:1 to 100:1.

In some of any of the respective embodiments described herein, a molar ratio of the agent (according to any of the respective embodiments described herein) to the DNase polypeptide contacted with the agent (according to any of the respective embodiments described herein) is at least 20:1. In some such embodiments, the molar ratio is from 20:1 to 10,000:1. In some embodiments, the molar ratio is from 20:1 to 5,000:1. In some embodiments, the molar ratio is from 20:1 to 2,000:1. In some embodiments, the molar ratio is from 20:1 to 1,000:1. In some embodiments, the molar ratio is from 20:1 to 500:1. In some embodiments, the molar ratio is from 20:1 to 200:1. In some embodiments, the molar ratio is from 20:1 to 100:1.

In some of any of the respective embodiments described herein, a molar ratio of the agent (according to any of the respective embodiments described herein) to the DNase polypeptide contacted with the agent (according to any of the respective embodiments described herein) is at least 50:1. In some such embodiments, the molar ratio is from 50:1 to 10,000:1. In some embodiments, the molar ratio is from 50:1 to 5,000:1. In some embodiments, the molar ratio is from 50:1 to 2,000:1. In some embodiments, the molar ratio is from 50:1 to 1,000:1. In some embodiments, the molar ratio is from 50:1 to 500:1. In some embodiments, the molar ratio is from 50:1 to 200:1. In some embodiments, the molar ratio is from 50:1 to 100:1.

In some of any of the respective embodiments described herein, a molar ratio of the agent (according to any of the respective embodiments described herein) to the DNase polypeptide contacted with the agent (according to any of the respective embodiments described herein) is at least 100:1. In some such embodiments, the molar ratio is from 100:1 to 10,000:1. In some embodiments, the molar ratio is from 100:1 to 5,000:1. In some embodiments, the molar ratio is from 100:1 to 2,000:1. In some embodiments, the molar ratio is from 50:1 to 1,000:1. In some embodiments, the molar ratio is from 100:1 to 500:1. In some embodiments, the molar ratio is from 100:1 to 200:1.

In some of any of the respective embodiments described herein, a molar ratio of the agent (according to any of the respective embodiments described herein) to the DNase polypeptide contacted with the agent (according to any of the respective embodiments described herein) is at least 200:1. In some such embodiments, the molar ratio is from 200:1 to 10,000:1. In some embodiments, the molar ratio is from 200:1 to 5,000:1. In some embodiments, the molar ratio is from 200:1 to 2,000:1. In some embodiments, the molar ratio is from 200:1 to 1,000:1. In some embodiments, the molar ratio is from 200:1 to 500:1.

In some of any of the respective embodiments described herein, a molar ratio of the agent (according to any of the respective embodiments described herein) to the DNase polypeptide contacted with the agent (according to any of the respective embodiments described herein) is at least 500:1. In some such embodiments, the molar ratio is from 500:1 to 10,000:1. In some embodiments, the molar ratio is from 500:1 to 5,000:1. In some embodiments, the molar ratio is from 500:1 to 2,000:1. In some embodiments, the molar ratio is from 500:1 to 1,000:1.

The molecular weight of the agent may optionally be selected to result in a poly(alkylene glycol) moiety having a molecular weight according to any of the embodiments described herein relating to poly(alkylene glycol) moiety molecular weight. The relationship between the molecular weights of a given agent and a poly(alkylene glycol) moiety generated from the agent in a process described herein will be apparent to the skilled person. For example, an agent of formula II will typically have a molecular weight which is 15 Da greater (e.g., essentially a rounding error for a molecular weight of 1 kDa or more) than a moiety of formula I (wherein the variables L₁, R₁, m and n are defined in the same manner, and L₂ is CH₂).

According to an aspect of some embodiments of the invention, there is provided a modified DNase protein obtainable according to the process described herein, in any of the respective embodiments.

Indications and Formulation:

The composition or modified DNase protein according to any of the respective embodiments described herein is optionally for use in treating a disease or disorder in which DNase activity is beneficial and/or for use in the treatment of a disease or disorder associated with excessive DNA (e.g., extracellular DNA, also referred to herein interchangeably as “cell-free DNA”) levels (e.g., in a fluid, secretion or tissue of a subject in need thereof).

According to an aspect of some embodiments described herein, there is provided a use of a modified DNase protein according to any of the respective embodiments described herein in the manufacture of a medicament for use in treating a disease or disorder in which DNase activity is beneficial and/or for use in the treatment of a disease or disorder associated with excessive DNA (e.g., extracellular DNA) levels (e.g., in a fluid, secretion or tissue of a subject in need thereof).

According to an aspect of some embodiments described herein, there is provided a method of treating a disease or disorder in which DNase activity is beneficial and/or a disease or disorder associated with excessive DNA (e.g., extracellular DNA) levels (e.g., in a fluid, secretion or tissue of a subject in need thereof), the method comprising administering to the subject a composition or modified DNase protein according to any of the respective embodiments described herein.

According to some of any of the respective embodiments of the invention (according to any of the aspects described herein), the disease or disorder is associated with DNA-related entities, such as neutrophil extracellular traps (NETs). The NETs may optionally be, for example, NETs associated with suicidal NETosis and/or vital NETosis.

Without being bound by any particular theory, it is believed that the modified DNase (according to any of the embodiments described herein) may exhibit a much longer half-life in vivo than the native human DNase, thus providing superior efficacy for the treatment of diseases or disorders characterized by the presence or accumulation of extracellular DNA, NETs and/or other DNA-related entities.

Treatment utilizing modified DNase according to any of the respective embodiments described herein may optionally be as monotherapy or by combination with current treatments, e.g., in combination with streptodornase for treatment of blood clot-related conditions.

Examples of diseases or disorders treatable according to embodiments of the invention include, without limitation, conditions associated with chronic neutrophilia (e.g., an increase in the number of neutrophils); neutrophil aggregation and leukostasis; thrombosis and vascular occlusion (e.g., sickle cell disease); ischemia-reperfusion injury (e.g., midgut volvulus, testicular torsion, limb ischemia reperfusion, vital organ ischemia-reperfusion, organ transplantation); surgical and traumatic tissue injury; an acute or chronic inflammatory reaction or disease; an autoimmune disease or disorder (e.g., systemic lupus erythematosus (SLE), lupus nephritis, rheumatoid arthritis, vasculitis, systemic sclerosis, psoriasis, atopic dermatitis, inflammatory bowel disease (IBD), ulcerative colitis, Crohn's disease, gout, rheumatoid arthritis, antiphospholipid syndrome); cardiovascular disease (e.g., myocardial infarction, stroke, atherosclerosis, venous thromboembolism, deep vein thrombosis (DVT), including thrombolytic therapy, coronary artery disease); a metabolic disease (e.g., diabetes); systemic inflammation (e.g., systemic inflammatory response syndrome (SIRS), sepsis, septicemia, septic shock, sepsis associated organ failure, disseminated intravascular coagulation (DIC), and thrombotic microangiopathy (TMA)); inflammatory diseases and disorders of the respiratory tract (e.g., cystic fibrosis, chronic obstructive pulmonary disease (COPD), acute lung injury (ALI), smoke-induced lung injury, transfusion-induced lung injury (TRALI), acute respiratory distress syndrome (ARDS), asthma, empyema, Kartegener's syndrome, lobar atelectasis, chronic bronchitis, bronchiectasis, primary ciliary dyskinesia, bronchiolitis, pleural infection); renal inflammatory diseases (acute and chronic kidney diseases, including acute kidney injury (AKI) and chronic kidney disease (CKD)); inflammatory diseases related to transplanted tissue (e.g., graft-versus-host disease); cancer (e.g., leukemia, tumor metastasis, and solid tumors, tumor metastasis following surgery, fibrosis and additional tissue damage associated with irradiation and/or chemotherapy treatment); a neurodegenerative disease or disorder; conditions associated with viral infection (e.g., conditions associated with COVID-19 and influenza, virus infection-associated sepsis, AKI, ALI or ARDS, and/or virus infection-associated thrombosis); and conditions associated with bacterial, fungal and/or protozoal infection (e.g., sepsis, AKI, ALI or ARDS, and/or thrombosis).

In some of any of the respective embodiments, the neurodegenerative disease or disorder is associated with an increased level of extracellular DNA (e.g., prokaryotic and/or human) in blood or cerebrospinal fluid or intestine of the patient, which level is higher than the control level (e.g., the level of extracellular DNA in blood or cerebrospinal fluid or intestine of a healthy age-matched individual or an average level of extracellular DNA in blood or cerebrospinal fluid or intestine of several healthy age-matched individuals). Non-limiting examples of neurodegenerative diseases and disorders include, e.g., Alzheimer's disease (e.g., late-onset Alzheimer's disease), Parkinson's disease, amyotrophic lateral sclerosis (ALS), Huntington's disease, and nervous system dysfunctions (e.g., schizophrenia or bipolar disorder).

Additional applications for modified DNase according to embodiments described herein include, without limitation, wound cleaning and promotion of wound healing, and treatment of ulcers (e.g., leg ulcers), post-pneumatic anemia, sinusitis, chronic hematomas, endocarditis, hepatorenal syndrome, hemothorax, intrabiliary blood clots, liver injury, liver infection, rhabdomyolysis, sarcoidosis, liver cirrhosis, fibrosis, female infertility, male infertility, heparin-induced thrombocytopenia, dry eye disease, acute coronary syndrome, and/or trauma (surgery, injury), for example, complications during cardiopulmonary bypass surgery, post-operative rhinoplasties.

Optionally, the modified DNase may be used to prevent or ameliorate neutropenia associated with chemotherapy, acute or chronic inflammatory disorder, or an acute or chronic infection.

In some embodiments, the subject has or is at risk of a ductal occlusion in a ductal system. Non-limiting examples of a ductal system or an organ or tissue containing a ductal system include bile duct, tear duct, lactiferous duct, cystic duct, hepatic duct, ejaculatory duct, parotid duct, submandibular duct, major sublingual duct, submandibular duct, Bartholin's duct, cerebral aqueduct, pancreas, mammary gland, vas deferens, ureter, urinary bladder, gallbladder, and liver. As such, the present invention is optionally useful for treating a subject who has pancreatitis, cholangitis, conjunctivitis, mastitis, dry eye disease, an obstruction of the vas deferens, or renal disease.

In other embodiments, the subject has or is at risk of NETs accumulating on endothelial surfaces (e.g., surgical adhesions), the skin (e.g., wounds/scarring, ulcers), or in synovial joints (e.g., gout, arthritis). For instance, NETs may contribute to surgical adhesions, e.g., after an invasive medical procedure. The present invention may optionally be administered during surgery to prevent or inhibit the formation of surgical adhesions.

In other embodiments, the modified DNase may be administered topically (e.g., to the skin) to prevent or treat wounds and/or scarring. Alternatively, the modified DNase may be administered to synovial joints to prevent or treat gout and arthritis.

In some embodiments, the composition is for use in treating a respiratory (e.g., pulmonary) condition and/or for reducing a viscosity (e.g., as represented by a reduction in a shear loss modulus and/or a shear storage modulus) of sputum. Respiratory conditions or diseases which can be treated by administration of modified DNase I protein according to any of the respective embodiments described herein include, without limitation, acute or chronic bronchopulmonary disease, atelectasis (e.g., due to tracheal or bronchial impaction and complications of tracheostomy), bronchitis or tracheobronchitis (e.g., chronic bronchitis, asthmatic bronchitis), cystic fibrosis, pneumonia, allergic diseases (e.g., allergic asthma), non-allergic asthma, tuberculosis, bronchopulmonary fungal infections, systemic lupus erythematosus, Sjogren's syndrome, bronchiectasis (e.g., non-cystic fibrosis bronchiectasis), emphysema, acute and chronic sinusitis, and the common cold.

In some embodiments of any of the embodiments described herein relating to a disease or disorder treatable by a DNase I activity, the disease or disorder is a suppurative disease or disorder. In some embodiments, the disease or disorder is a suppurative lung disease. In some embodiments, the disease or disorder is a chronic suppurative lung disease (CSLD), e.g., a disease or disorder characterized by a chronic wet cough and progressive lung damage. A CSLD treatable according to embodiments of the invention may optionally be cystic fibrosis or a non-cystic fibrosis CSLD. Examples of a non-cystic fibrosis CSLD include, without limitation, non-cystic fibrosis bronchiectasis, and chronic obstructive pulmonary disorder (COPD) (including chronic bronchitis and emphysema). In some embodiments, the disease or disorder is cystic fibrosis.

Without being bound by any particular theory, it is believed that the longer half-life of modified DNase proteins described herein may be particularly useful in applications involving systemic treatment, in which clearance of non-modified DNase represents a major obstacle to its utility for treatment, and treatment of conditions in which systemic administration may be beneficial; whereas non-modified DNase administered to the respiratory tract is less affected by a short half-life which can be overcome by poly(alkylene glycol) moieties (e.g., due to less rapid clearance of proteins in the respiratory tract).

In some embodiments of any of the embodiments described herein relating to a treatment, the subject to be treated is afflicted by a Pseudomonas (e.g., Pseudomonas aeruginosa) lung infections, optionally in addition to a pulmonary disease or condition described herein, such as cystic fibrosis.

The modified DNase protein according to any one of the respective embodiments described herein can be used to produce a pharmaceutical composition, and/or used and/or administered in essentially the same manner as described in International Patent Application Publication WO 2016/108244 (according to any of the embodiments described therein), the contents of which are incorporated herein in their entirety, particularly contents regarding pharmaceutical compositions, uses of modified DNase I and pulmonary administration. For example, administration may be systemic or local; and/or via inhalation, topical and/or or via injection.

It is expected that during the life of a patent maturing from this application many relevant conditions associated with DNA and/or NETs will be uncovered and the scope of the term “treating” and grammatical variants thereof is intended to include all such new technologies a priori.

The modified DNase protein according to any of the respective embodiments described herein may optionally be used per se, or alternatively, as part of a pharmaceutical composition which further comprises a pharmaceutically acceptable carrier.

As used herein a “pharmaceutical composition” refers to a preparation of one or more species of modified DNase described herein, with other chemical components such as pharmaceutically acceptable and suitable carriers and excipients. The purpose of a pharmaceutical composition is to facilitate administration of a compound to an organism.

Hereinafter, the term “pharmaceutically acceptable carrier” refers to a carrier or a diluent that does not cause significant irritation to an organism and does not abrogate the biological activity and properties of the administered compound. Examples, without limitations, of carriers are propylene glycol, saline, emulsions and mixtures of organic solvents with water, as well as solid (e.g., powdered) and gaseous carriers.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polymers such as polyethylene glycols.

Techniques for formulation and administration of drugs may be found in “Remington's Pharmaceutical Sciences” Mack Publishing Co., Easton, PA, latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more pharmaceutically acceptable carriers comprising excipients and auxiliaries, which facilitate processing of the modified DNase protein into preparations which can be used pharmaceutically. Proper formulation is dependent upon the route of administration chosen.

The modified DNase protein described herein may be formulated for parenteral administration, e.g., by bolus injection or continuous infusion. Formulations for injection or infusion may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the modified DNase preparation in water-soluble form. For injection or infusion, the modified DNase may optionally be formulated in aqueous solutions, preferably in physiologically compatible buffers such as Hank's solution, Ringer's solution, or physiological saline buffer with or without organic solvents such as propylene glycol, polyethylene glycol.

Additionally, suspensions of the modified DNase protein may be prepared as appropriate oily injection suspensions and emulsions (e.g., water-in-oil, oil-in-water or water-in-oil in oil emulsions). Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the modified DNase protein to allow for the preparation of highly concentrated solutions.

Injection and/or infusion directly into the blood stream (e.g., intravenous administration) may be a particularly suitable for treating an elevated level of extracellular DNA and/or NETs in the blood (including any condition associated therewith). Administration into the bloodstream may optionally also be used to deliver the modified DNase protein to a particular tissue.

Alternatively or additionally, the modified DNase protein may be injected locally, e.g., to a tissue afflicted by elevated levels of extracellular DNA and/or NETs. The tissue is optionally a tissue associated with an inflammation.

For transmucosal administration, penetrants are used in the formulation. Such penetrants are generally known in the art.

For oral administration, the modified DNase protein of the invention can be formulated readily by combining the modified DNase protein with pharmaceutically acceptable carriers well known in the art. Such carriers enable the modified DNase protein described herein to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethylcellulose, sodium carboxymethylcellulose; and/or physiologically acceptable polymers such as polyvinylpyrrolidone (PVP). If desired, disintegrating agents may be added, such as cross-linked polyvinylpyrrolidone, agar, or alginic acid or a salt thereof such as sodium alginate.

Dragee cores are provided with suitable coatings. For this purpose, concentrated sugar solutions may be used which may optionally contain gum arabic, talc, polyvinylpyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of doses of active modified DNase protein.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the modified DNase protein may be dissolved or suspended in suitable liquids, such as fatty oils, liquid paraffin, or liquid polyethylene glycols. In addition, stabilizers may be added. All formulations for oral administration should be in dosages suitable for the chosen route of administration.

The modified DNase protein of embodiments of the present invention may also be formulated in rectal compositions such as suppositories or retention enemas, using, e.g., conventional suppository bases such as cocoa butter or other glycerides.

Oral and/or rectal administration may be a particularly suitable for treating a disease or disorder of the gastrointestinal tract, for example, a condition associated with inflammation of the gastrointestinal tract (e.g., inflammatory bowel disease and/or gastroenteritis).

For buccal administration, the compositions may take the form of tablets or lozenges formulated in conventional manner.

For administration by inhalation (e.g., for treating a pulmonary disease or disorder, or to effect systemic administration), the pharmaceutical compositions may optionally be, for example, a propellant-containing aerosol (e.g., with dichlorodifluoromethane, trichlorofluoromethane, dichloro-tetrafluoroethane or carbon dioxide propellant), or a propellant-free inhalable solution or suspension. In some embodiments, the composition is a propellant-free inhalable solution comprising the modified DNase, which is suitable for being administered to the subject, for example, via a nebulizer. Other suitable preparations include, but are not limited to, mist, vapor, or spray preparations so long as the particles comprising the protein composition are delivered in a size range consistent with that described for the delivery device, e.g., a dry powder form of the pharmaceutical composition. In some embodiments, the composition is formulated for delivery via a nebulizer.

The modified DNase protein is optionally conveniently delivered in the form of an aerosol spray presentation (which typically includes powdered, liquefied and/or gaseous carriers) from a pressurized pack or a nebulizer, with the use of a suitable propellant. In the case of a pressurized aerosol, the dosage unit may be determined by providing a valve to deliver a metered amount. Capsules and cartridges of, e.g., gelatin for use in an inhaler or insufflator may be formulated containing a powder mix of the modified DNase protein and a suitable powder base such as, but not limited to, lactose or starch.

Where a liquid solution or suspension is used in a delivery device, a nebulizer, a metered dose inhaler, or other suitable delivery device delivers, in a single or multiple fractional dose, by pulmonary inhalation, a pharmaceutically effective amount of the composition to the subject's lungs as droplets, e.g., having the same particle size range described herein. Methods for preparing and using formulations suitable for use as liquid or suspension are known in the art, for example, the oil-based matrix taught in International Patent Application Publication WO 2011/004476.

Where the liquid pharmaceutical composition is lyophilized prior to use in the delivery methods of the invention, the lyophilized composition may be milled to obtain the finely divided dry powder consisting of particles within the desired size range described herein. Where spray-drying is used to obtain a dry powder form of the liquid pharmaceutical composition, the process is carried out under conditions that result in a substantially amorphous finely divided dry powder consisting of particles within the desired size range noted above. Similarly, if the starting pharmaceutical composition is already in a lyophilized form, the composition can be milled to obtain the dry powder form for subsequent preparation as an aerosol or other preparation suitable for pulmonary inhalation. Where the starting pharmaceutical composition is in its spray-dried form, the composition has preferably been prepared such that it is already in a dry powder form having the appropriate particle size for dispensing as an aqueous or non-aqueous solution or suspension in accordance with the pulmonary administration methods of the invention. For methods of preparing dry powder forms of pharmaceutical compositions, see, for example, International Patent Application Publications WO 96/32149, WO 97/41833 and WO 98/29096, and U.S. Pat. Nos. 5,976,574, 5,985,248, and 6,001,336, herein incorporated by reference.

The resulting dry powder form of the composition is then optionally placed within an appropriate delivery device for subsequent preparation as an aerosol or other suitable preparation that is delivered to the subject via pulmonary inhalation.

Where the dry powder form of the pharmaceutical composition is to be prepared and dispensed as an aqueous or non-aqueous solution or suspension, a metered-dose inhaler, or other appropriate delivery device is optionally used.

The dry powder form of the pharmaceutical composition according to some embodiments of the invention may optionally be reconstituted to an aqueous solution for subsequent delivery as an aqueous solution aerosol using a nebulizer, a metered dose inhaler, or other suitable delivery device. In the case of a nebulizer, the aqueous solution held within a fluid reservoir is converted into an aqueous spray, only a small portion of which leaves the nebulizer for delivery to the subject at any given time.

The remaining spray drains back into a fluid reservoir within the nebulizer, where it is aerosolized again into an aqueous spray. This process is repeated until the fluid reservoir is completely dispensed or until administration of the aerosolized spray is terminated. Examples of nebulizers are described herein.

Alternatively, the modified DNase protein may be in powder form for constitution with a suitable vehicle, e.g., sterile, pyrogen-free water, before use.

Pharmaceutical compositions suitable for use in the context of the present invention include compositions wherein the active ingredients are contained in an amount effective to achieve the intended purpose. More specifically, a therapeutically effective amount means an amount of modified DNase protein effective to prevent, alleviate or ameliorate symptoms of disease or prolong the survival of the subject being treated.

For any modified DNase protein used according to embodiments the invention, the therapeutically effective amount or dose can be estimated initially from activity assays in animals. For example, a dose can be formulated in animal models to achieve a circulating concentration range that includes the IC₅₀ as determined by activity assays (e.g., the concentration of the test protein structures, which achieves a half-maximal increase in a biological activity of the modified DNase protein). Such information can be used to more accurately determine useful doses in humans.

As is demonstrated in the Examples section that follows, a therapeutically effective amount for the modified DNase protein of embodiments of the present invention may range between about 0.1 μg/kg body weight and about 500 mg/kg body weight.

Toxicity and therapeutic efficacy of the modified DNase protein described herein can be determined by standard pharmaceutical procedures in experimental animals, e.g., by determining the EC₅₀, the IC₅₀ and the LD₅₀ (lethal dose causing death in 50% of the tested animals) for a subject protein structure. The data obtained from these activity assays and animal studies can be used in formulating a range of dosage for use in humans.

The dosage may vary depending upon the dosage form employed and the route of administration utilized. The exact formulation, route of administration and dosage can be chosen by the individual physician in view of the patient's condition. (See e.g., Fingl et al., 1975, in “The Pharmacological Basis of Therapeutics”, Ch. 1 p. 1).

Dosage amount and interval may be adjusted individually to provide plasma levels of the active DNase which are sufficient to maintain the desired effects, termed the minimal effective concentration (MEC). The MEC will vary for each preparation, but can be estimated from in vitro data; e.g., the concentration necessary to achieve the desired level of activity in vitro. Dosages necessary to achieve the MEC will depend on individual characteristics and route of administration. HPLC assays or bioassays can be used to determine plasma concentrations.

Dosage intervals can also be determined using the MEC value. Preparations should be administered using a regimen, which maintains plasma levels above the MEC for 10-90% of the time, preferably between 30-90% and most preferably 50-90%.

As discussed herein, modified DNase protein described herein may exhibit a long half-life in the body. Such a property may allow the use of relatively infrequent administration (which may be particularly advantageous when administration is by an inconvenient route such as injection) and/or administration of relatively low doses (which may be particularly advantageous for decreasing toxicity and/or a potential immune response to the modified DNase protein).

In some of any of the embodiments described herein, an administration frequency and dose per administration are selected such that the administered dosage of modified DNase protein (e.g., by injection to an adult human subject) is no more than 200 mg modified DNase protein per month (for example, administration of 600 mg at intervals of 3 months would be considered a dosage of 20 mg per month). In some such embodiments, the dosage is no more than 100 mg per month. In some embodiments, the dosage is no more than 50 mg per month. In some embodiments, the dosage is no more than 20 mg per month. In some embodiments, the dosage is no more than 10 mg per month. In some embodiments, the dosage is no more than 5 mg per month. In some embodiments, the dosage is no more than 2 mg per month. In some embodiments, the dosage is no more than 1 mg per month.

Depending on the severity and responsiveness of the condition to be treated, dosing can also be a single administration, optionally of a slow release composition described hereinabove, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA (the U.S. Food and Drug Administration) approved kit, which may contain one or more unit dosage forms containing the active ingredient. The pack may, for example, comprise metal or plastic foil, such as, but not limited to a blister pack or a pressurized container (for inhalation). The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions comprising a modified DNase protein of any of the embodiments of the invention formulated in a compatible pharmaceutical carrier may also be prepared, placed in an appropriate container, and labeled for treatment of an indicated condition or diagnosis, as is detailed herein.

Thus, according to an embodiment of the present invention, the pharmaceutical composition described herein is packaged in a packaging material and identified in print, in or on the packaging material, for use in the treatment of a condition in which the activity of the modified DNase protein is beneficial, as described hereinabove.

### Additional Definitions

Herein, the terms “hydrocarbon” and “hydrocarbon moiety” describe an organic moiety that includes, as its basic skeleton, a chain of carbon atoms, substituted mainly by hydrogen atoms. The hydrocarbon can be saturated or non-saturated, be comprised of aliphatic, alicyclic or aromatic moieties, and can optionally be substituted by one or more substituents (other than hydrogen). A substituted hydrocarbon may have one or more substituents, whereby each substituent group can independently be, for example, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. The hydrocarbon can be an end group or a linking group, as these terms are defined herein. Preferably, the hydrocarbon moiety has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. Optionally, the hydrocarbon is a medium size hydrocarbon having 1 to 10 carbon atoms. Optionally, the hydrocarbon has 1 to 4 carbon atoms.

As used herein throughout, the term “alkyl” refers to any saturated aliphatic hydrocarbon including straight chain and branched chain groups. Preferably, the alkyl group has 1 to 20 carbon atoms. Whenever a numerical range; e.g., “1 to 20”, is stated herein, it implies that the group, in this case the hydrocarbon, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted. When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted. Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted. Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino.

The term “alkylene” describes a saturated or unsaturated aliphatic hydrocarbon linking group, as this term is defined herein, which differs from an alkyl group (when saturated) or an alkenyl or alkynyl group (when unsaturated), as defined herein, only in that alkylene is a linking group rather than an end group (as these terms are defined herein).

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond.

An “aryl” group refers to an all-carbon monocyclic or fused-ring polycyclic (i.e., rings which share adjacent pairs of carbon atoms) having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroaryl” group refers to a monocyclic or fused ring (i.e., rings which share an adjacent pair of atoms) having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, imine, oxime, hydrazone, carbonyl, thiocarbonyl, a urea group, a thiourea group, O-carbamyl, N-carbamyl, O-thiocarbamyl, N-thiocarbamyl, S-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like.

Herein, the terms “amine” and “amino” each refer to either a —NR′R″ group or a —N⁺R′R″R′″ group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is not substituted by oxo (unless explicitly indicated otherwise), such that R′, R″ and R′″ are not (for example) carbonyl, C-carboxy or amide, as these groups are defined herein.

An “azide” group refers to a —N═N⁺═N⁻ group.

An “alkoxy” group refers to any of an —O-alkyl, —O-alkenyl, —O-alkynyl, —O-cycloalkyl, and —O-heteroalicyclic group, as defined herein.

An “aryloxy” group refers to both an —O-aryl and an —O-heteroaryl group, as defined herein.

A “hydroxy” group refers to a —OH group.

A “thiohydroxy” or “thiol” group refers to a —SH group.

A “thioalkoxy” group refers to any of an —S-alkyl, —S-alkenyl, —S-alkynyl, —S-cycloalkyl, and —S-heteroalicyclic group, as defined herein.

A “thioaryloxy” group refers to both an —S-aryl and an —S-heteroaryl group, as defined herein.

A “carbonyl” or “acyl” group refers to a —C(═O)—R′ group, where R′ is defined as hereinabove.

A “thiocarbonyl” group refers to a —C(═S)—R′ group, where R′ is as defined herein.

A “C-carboxy” group refers to a —C(═O)—O—R′ group, where R′ is as defined herein.

An “O-carboxy” group refers to an R′C(═O)—O— group, where R′ is as defined herein.

A “carboxylic acid” group refers to a —C(═O)OH group.

An “oxo” group refers to a ═O group.

An “imine” group refers to a ═N—R′ group, where R′ is as defined herein.

An “oxime” group refers to a ═N—OH group.

A “hydrazone” group refers to a ═N—NR′R″ group, where each of R′ and R″ is as defined herein.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R'S(═O)₂—NR″— group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″— group, where each of R′ and R″ is as defined herein.

An “O-thiocarbamyl” group refers to an —OC(═S)—NR′R″ group, where each of R′ and R″ is as defined herein.

An “N-thiocarbamyl” group refers to an R′OC(═S)NR″— group, where each of R′ and R″ is as defined herein.

An “S-thiocarbamyl” group refers to an —SC(═O)—NR′R″ group, where each of R′ and R″ is as defined herein.

An “amide” or “amido” group encompasses C-amido and N-amido groups, as defined herein.

A “C-amido” group refers to a —C(═O)—NR′R″ group, where each of R′ and R″ is as defined herein.

An “N-amido” group refers to an R′C(═O)—NR″— group, where each of R′ and R″ is as defined herein.

A “urea group” refers to an —N(R′)—C(═O)—NR″R′″ group, where each of R′, R″ and R″ is as defined herein.

A “thiourea group” refers to a —N(R′)—C(═S)—NR″R′″ group, where each of R′, R″ and R″ is as defined herein.

A “nitro” group refers to an —NO₂ group.

A “cyano” group refers to a —C≡N group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

The term “phosphinyl” describes a —PR′R″ group, with each of R′ and R″ as defined hereinabove.

The term “hydrazine” describes a —NR′—NR″R′″ group, with R′, R″, and R′″ as defined herein.

As used herein, the term “hydrazide” describes a —C(═O)—NR′—NR″R′″ group, where R′, R″ and R′ are as defined herein.

As used herein, the term “thiohydrazide” describes a —C(═S)—NR′—NR″R′″ group, where R′, R″ and R′″ are as defined herein.

A “guanidinyl” group refers to an —RaNC(═NRd)-NRbRc group, where each of Ra, Rb, Rc and Rd can be as defined herein for R′ and R″.

A “guanyl” or “guanine” group refers to an RaRbNC(═NRd)- group, where Ra, Rb and Rd are as defined herein.

The compounds and structures described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

For any of the embodiments described herein, the compound described herein may be in a form of a salt, for example, a pharmaceutically acceptable salt, and/or in a form of a prodrug.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

As used herein, the term “prodrug” refers to a compound which is converted in the body to an active compound (e.g., the compound of the formula described hereinabove). A prodrug is typically designed to facilitate administration, e.g., by enhancing absorption. A prodrug may comprise, for example, the active compound modified with ester groups, for example, wherein any one or more of the hydroxyl groups of a compound is modified by an acyl group, optionally (C₁₋₄)-acyl (e.g., acetyl) group to form an ester group, and/or any one or more of the carboxylic acid groups of the compound is modified by an alkoxy or aryloxy group, optionally (C₁₋₄)-alkoxy (e.g., methyl, ethyl) group to form an ester group.

Further, each of the compounds described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The compounds described herein can be used as polymorphs and the present embodiments further encompass any isomorph of the compounds and any combination thereof.

Herein, the term “polypeptide” refers to a polymer comprising at least 10 amino acid residues linked by peptide bonds or analogs thereof (as described herein below), and optionally only by peptide bonds per se. In some embodiments, the polypeptide comprises at least 20 amino acid residues or analogs thereof. In some embodiments, the polypeptide comprises at least 30 amino acid residues or analogs thereof. In some embodiments, the polypeptide comprises at least 50 amino acid residues or analogs thereof.

The term “polypeptide” encompasses native polypeptides (e.g., degradation products, synthetically synthesized polypeptides and/or recombinant polypeptides), including, without limitation, native proteins, fragments of native proteins and homologs of native proteins and/or fragments thereof; as well as peptidomimetics (typically, synthetically synthesized polypeptides) and peptoids and semipeptoids which are polypeptide analogs, which may have, for example, modifications rendering the polypeptides more stable while in a body or more capable of penetrating into cells. Such modifications include, but are not limited to N-terminus modification, C-terminus modification, peptide bond modification, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided herein below.

Peptide bonds (—CO—NH—) within the polypeptide may be substituted, for example, by N-methylated amide bonds (—N(CH3)-CO—), ester bonds (—C(═O)—O—), ketomethylene bonds (—CO—CH2-), sulfinylmethylene bonds (—S(═O)—CH2-), α-aza bonds (—NH—N(R)—CO—), wherein R is any alkyl (e.g., methyl), amine bonds (—CH2-NH—), sulfide bonds (˜CH2-S—), ethylene bonds (˜CH2-CH2-), hydroxyethylene bonds (—CH(OH)—CH2-), thioamide bonds (—CS—NH—), olefinic double bonds (—CH═CH—), fluorinated olefinic double bonds (—CF═CH—), retro amide bonds (—NH—CO—), peptide derivatives (—N(R)—CH2-CO—), wherein R is the “normal” side chain, naturally present on the carbon atom.

These modifications can occur at any of the bonds along the polypeptide chain and even at several (2-3) bonds at the same time.

Natural aromatic amino acids, Trp, Tyr and Phe, may be substituted by non-natural aromatic amino acids such as 1,2,3,4-tetrahydroisoquinoline-3-carboxylic acid (Tic), naphthylalanine, ring-methylated derivatives of Phe, halogenated derivatives of Phe or O-methyl-Tyr.

The polypeptides of some embodiments of the invention may also include one or more modified amino acids or one or more non-amino acid monomers (e.g. fatty acids, complex carbohydrates etc.).

The term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1), and non-conventional or modified amino acids (e.g., synthetic, Table 2) which can be used with some embodiments of the invention.

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional | | Non-conventional | |
|---|---|---|---|
| amino acid | Code | amino acid | Code |
| ornithine | Orn | hydroxyproline | Hyp |
| α-aminobutyric acid | Abu | aminonorbornyl-carboxylate | Norb |
| D-alanine | Dala | aminocyclopropane-carboxylate | Cpro |
| D-arginine | Darg | N-(3-guanidinopropyl)glycine | Narg |
| D-asparagine | Dasn | N-(carbamylmethyl)glycine | Nasn |
| D-aspartic acid | Dasp | N-(carboxymethyl)glycine | Nasp |
| D-cysteine | Dcys | N-(thiomethyl)glycine | Ncys |
| D-glutamine | Dgln | N-(2-carbamylethyl)glycine | Ngln |
| D-glutamic acid | Dglu | N-(2-carboxyethyl)glycine | Nglu |
| D-histidine | Dhis | N-(imidazolylethyl)glycine | Nhis |
| D-isoleucine | Dile | N-(1-methylpropyl)glycine | Nile |
| D-leucine | Dleu | N-(2-methylpropyl)glycine | Nleu |
| D-lysine | Dlys | N-(4-aminobutyl)glycine | Nlys |
| D-methionine | Dmet | N-(2-methylthioethyl)glycine | Nmet |
| D-ornithine | Dorn | N-(3-aminopropyl)glycine | Norn |
| D-phenylalanine | Dphe | N-benzylglycine | Nphe |
| D-proline | Dpro | N-(hydroxymethyl)glycine | Nser |
| D-serine | Dser | N-(1-hydroxyethyl)glycine | Nthr |
| D-threonine | Dthr | N-(3-indolylethyl) glycine | Nhtrp |
| D-tryptophan | Dtrp | N-(p-hydroxyphenyl)glycine | Ntyr |
| D-tyrosine | Dtyr | N-(1-methylethyl)glycine | Nval |
| D-valine | Dval | N-methylglycine | Nmgly |
| D-N-methylalanine | Dnmala | L-N-methylalanine | Nmala |
| D-N-methylarginine | Dnmarg | L-N-methylarginine | Nmarg |
| D-N-methylasparagine | Dnmasn | L-N-methylasparagine | Nmasn |
| D-N-methylasparatate | Dnmasp | L-N-methylaspartic acid | Nmasp |
| D-N-methylcysteine | Dnmcys | L-N-methylcysteine | Nmcys |
| D-N-methylglutamine | Dnmgln | L-N-methylglutamine | Nmgln |
| D-N-methylglutamate | Dnmglu | L-N-methylglutamic acid | Nmglu |
| D-N-methylhistidine | Dnmhis | L-N-methylhistidine | Nmhis |
| D-N-methylisoleucine | Dnmile | L-N-methylisolleucine | Nmile |
| D-N-methylleucine | Dnmleu | L-N-methylleucine | Nmleu |
| D-N-methyllysine | Dnmlys | L-N-methyllysine | Nmlys |
| D-N-methylmethionine | Dnmmet | L-N-methylmethionine | Nmmet |
| D-N-methylornithine | Dnmorn | L-N-methylornithine | Nmorn |
| D-N-methylphenylalanine | Dnmphe | L-N-methylphenylalanine | Nmphe |
| D-N-methylproline | Dnmpro | L-N-methylproline | Nmpro |
| D-N-methylserine | Dnmser | L-N-methylserine | Nmser |
| D-N-methylthreonine | Dnmthr | L-N-methylthreonine | Nmthr |
| D-N-methyltryptophan | Dnmtrp | L-N-methyltryptophan | Nmtrp |
| D-N-methyltyrosine | Dnmtyr | L-N-methyltyrosine | Nmtyr |
| D-N-methylvaline | Dnmval | L-N-methylvaline | Nmval |
| L-norleucine | Nle | L-N-methylnorleucine | Nmnle |
| L-norvaline | Nva | L-N-methylnorvaline | Nmnva |
| L-ethylglycine | Etg | L-N-methyl-ethylglycine | Nmetg |
| L-t-butylglycine | Tbug | L-N-methyl-t-butylglycine | Nmtbug |
| L-homophenylalanine | Hphe | L-N-methyl-homophenylalanine | Nmhphe |
| α-naphthylalanine | Anap | N-methyl-α-naphthylalanine | Nmanap |
| penicillamine | Pen | N-methylpenicillamine | Nmpen |
| γ-aminobutyric acid | Gabu | N-methyl-γ-aminobutyrate | Nmgabu |
| cyclohexylalanine | Chexa | N-methyl-cyclohexylalanine | Nmchexa |
| cyclopentylalanine | Cpen | N-methyl-cyclopentylalanine | Nmcpen |
| α-amino-α-methylbutyrate | Aabu | N-methyl-α-amino-α-methylbutyrate | Nmaabu |
| α-aminoisobutyric acid | Aib | N-methyl-α-aminoisobutyrate | Nmaib |
| D-α-methylarginine | Dmarg | L-α-methylarginine | Marg |
| D-α-methylasparagine | Dmasn | L-α-methylasparagine | Masn |
| D-α-methylaspartate | Dmasp | L-α-methylaspartate | Masp |
| D-α-methylcysteine | Dmcys | L-α-methylcysteine | Mcys |
| D-α-methylglutamine | Dmgln | L-α-methylglutamine | Mgln |
| D-α-methyl glutamic acid | Dmglu | L-α-methylglutamate | Mglu |
| D-α-methylhistidine | Dmhis | L-α-methylhistidine | Mhis |
| D-α-methylisoleucine | Dmile | L-α-methylisoleucine | Mile |
| D-α-methylleucine | Dmleu | L-α-methylleucine | Mleu |
| D-α-methyllysine | Dmlys | L-α-methyllysine | Mlys |
| D-α-methylmethionine | Dmmet | L-α-methylmethionine | Mmet |
| D-α-methylornithine | Dmorn | L-α-methylornithine | Morn |
| D-α-methylphenylalanine | Dmphe | L-α-methylphenylalanine | Mphe |
| D-α-methylproline | Dmpro | L-α-methylproline | Mpro |
| D-α-methylserine | Dmser | L-α-methylserine | Mser |
| D-α-methylthreonine | Dmthr | L-α-methylthreonine | Mthr |
| D-α-methyltryptophan | Dmtrp | L-α-methyltryptophan | Mtrp |
| D-α-methyltyrosine | Dmtyr | L-α-methyltyrosine | Mtyr |
| D-α-methylvaline | Dmval | L-α-methylvaline | Mval |
| N-cyclobutylglycine | Ncbut | L-α-methylnorvaline | Mnva |
| N-cycloheptylglycine | Nchep | L-α-methylethylglycine | Metg |
| N-cyclohexylglycine | Nchex | L-α-methyl-t-butylglycine | Mtbug |
| N-cyclodecylglycine | Ncdec | L-α-methyl-homophenylalanine | Mhphe |
| N-cyclododecylglycine | Ncdod | α-methyl-α-naphthylalanine | Manap |
| N-cyclooctylglycine | Ncoct | α-methylpenicillamine | Mpen |
| N-cyclopropylglycine | Ncpro | α-methyl-γ-aminobutyrate | Mgabu |
| N-cycloundecylglycine | Ncund | α-methyl-cyclohexylalanine | Mchexa |
| N-(2-aminoethyl)glycine | Naeg | α-methyl-cyclopentylalanine | Mcpen |
| N-(2,2-diphenylethyl)glycine | Nbhm | N-(N-(2,2-diphenylethyl) | Nnbhm |
| | | carbamylmethyl-glycine | |
| N-(3,3-diphenylpropyl)glycine | Nbhe | N-(N-(3,3-diphenylpropyl) | Nnbhe |
| | | carbamylmethyl-glycine | |
| 1-carboxy-1-(2,2-diphenyl | Nmbc | 1,2,3,4-tetrahydroisoquinoline- | Tic |
| ethylamino)cyclopropane | | 3-carboxylic acid | |
| phosphoserine | pSer | phosphothreonine | pThr |
| phosphotyrosine | pTyr | O-methyl-tyrosine | |
| 2-aminoadipic acid | | hydroxylysine | |

The polypeptides of some embodiments of the invention are preferably utilized in a linear form, although it will be appreciated that in cases where cyclization does not severely interfere with polypeptide characteristics, cyclic forms of the polypeptide can also be utilized.

Since the present polypeptides are preferably utilized in therapeutics or diagnostics which require the polypeptides to be in soluble form, the polypeptides of some embodiments of the invention preferably include one or more non-natural or natural polar amino acids, including but not limited to serine and threonine which are capable of increasing polypeptide solubility due to their hydroxyl-containing side chain.

The polypeptides of some embodiments of the invention may be synthesized by any techniques that are known to those skilled in the art of peptide synthesis. For solid phase peptide synthesis, a summary of the many techniques may be found in J. M. Stewart and J. D. Young, Solid Phase Peptide Synthesis, W. H. Freeman Co. (San Francisco), 1963 and J. Meienhofer, Hormonal Proteins and Peptides, vol. 2, p. 46, Academic Press (New York), 1973. For classical solution synthesis see G. Schroder and K. Lupke, The Peptides, vol. 1, Academic Press (New York), 1965.

In general, these methods comprise the sequential addition of one or more amino acids or suitably protected amino acids to a growing peptide chain. Normally, either the amino or carboxyl group of the first amino acid is protected by a suitable protecting group. The protected or derivatized amino acid can then either be attached to an inert solid support or utilized in solution by adding the next amino acid in the sequence having the complimentary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups (and any solid support) are removed sequentially or concurrently, to afford the final polypeptide compound. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide and so forth. Further description of peptide synthesis is disclosed in U.S. Pat. No. 6,472,505.

A preferred method of preparing the polypeptide compounds of some embodiments of the invention involves solid phase peptide synthesis.

Large scale peptide synthesis is described by Andersson [Biopolymers 2000, 55(3):227-50].

As used herein the term “about” refers to ±20%, and in optional embodiments refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); Hermanson, “Bioconjugate Techniques”, 2nd Edition, Elsevier Inc., Burlington, M A (2008); methodologies as set forth in U.S. Pat. Nos. 4,666,828; 4,683,202; 4,801,531; and U.S. Pat. No. 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, C A (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Materials:

Sodium cyanoborohydride (NaBH₃CN), Methyl Green, buffer components, CaCl₂, MgCl₂ and other chemicals were obtained from Sigma-Aldrich.

Monofunctional polyethylene glycol propionaldehyde (PEG-Ald) reagents were obtained from Creative PEGWorks, NOF, and JenKem Technology USA Inc.

Polyethylene glycol bis-N-hydroxysuccinimide bis-NHS-PEG reagents were obtained from Rapp Polymere GmbH and Iris Biotech GmbH.

Plant Recombinant Human DNase I:

Plant recombinant human DNase I was prepared as described in International Patent Application Publication WO 2013/114374, by being expressed in transgenic Nicotiana tabacum Bright Yellow-2 (BY2) cell culture and purified from the extracellular media. The DNase I generally contained a mixture of amino acid sequences, in which the majority had SEQ ID NO: 1, and a small fraction had SEQ ID NO: 2.

BY2 suspension culture was co-cultivated, for 48 hours, with the Agrobacterium tumefaciens EHA105 strain carrying the vector harboring the DNase I gene and the neomycin phosphotransferase (NPTII) selection gene.

Subsequently, the cells were kept in medium supplemented with 50 mg/L of kanamycin and 250 mg/L cefotaxime. The NPTII gene confers resistance to kanamycin—thus, only NPTII positive BY2 cells survive in this selection medium. The cefotaxime was used to selectively kill the Agrobacterium, the plant cells being resistant to this antibiotic. Once a nicely growing transgenic cell suspension was established, it was used for screening and isolating individual cell lines. To allow for the selection of individual cell lines, aliquots of highly diluted cell suspension were spread on solid BY2 medium. The cells were then grown until small calli developed. Each callus was then re-suspended in liquid culture. Medium was then sampled and evaluated for DNase I levels. The lines that secreted relatively high DNase I levels were then further re-analyzed and compared for DNase I levels, ending with the final selection of candidate DNase I expressing lines.

Media samples of transformed BY2 cells expressing the human DNase I protein were collected and when required, concentrated ×5 by Amicon™ Ultra centrifugal filters (10 kDa cutoff). DNase I catalytic activity in cell media was determined by DNA-methyl green assay and compared to total DNase I amount, as determined by enzyme-linked immunosorbent assay.

The plant recombinant human DNase I (prh-DNase I) was purified using four chromatographic steps, including ion exchange and hydrophobic interactions, and two ultra-filtration steps. A highly pure prh-DNase I was obtained at a concentration of 5 mg/mL.

Reaction with PEG-NHS

DNase I was diluted in MES buffer (100 mM, pH 7), and CaCl₂ was added to the reaction mixture. PEG-N-hydroxysuccinimde (PEG-NHS) was added and the reaction mixture was gently agitated for 2 hours at room temperature. A molar ratio of 1:200 of DNase I to PEG-NHS was used in the reaction. The final concentrations were 2 mg/mL protein and 10 mM CaCl₂. The reaction was stopped by dialysis, using an Amicon™ filter with a 30 kDa cutoff (Merck), to formulation buffer.

Reaction of DNase with PEG-Propionaldehyde (PEG-Ald)

DNase I was added to PEG-propionaldehyde (PEG-Ald) diluted in MES (2-(N-morpholino)ethanesulfonic acid) buffer (100 mM, pH 7), and CaCl₂ was added to the reaction mixture, following by addition of freshly prepared NaBH₃CN in MES buffer (100 mM, pH 7). The molar ratio used in reaction was calculated against protein and ranged from 1:100 to 1:600 (protein:PEG-Ald). The final concentrations were 2 mg/mL protein, 10 mM CaCl₂, and 100 mM NaBH₃CN. The reaction was overnight (at least 10 hours) at room temperature with gentle agitation. The reaction was stopped by dialysis, using an Amicon™ filter with a 30 kDa cutoff (Merck), to formulation or loading buffers.

Optical Density

The quantitation of purified proteins was obtained from their absorbance at 280 nm (extinction coefficient of 1.43 cm⁻¹ (gr/liter)⁻¹), using a NanoDrop™ 2000 apparatus (Thermo Fisher Scientific).

Assessment of Protein Content and Activity by Methyl Green-Based Activity Assay:

Activity of DNase I and modified DNase I species was assessed by a methyl green enzymatic activity assay, employing DNA from salmon testis complexed with methyl green as a substrate. The dye methyl green intercalates between the stacked bases of double-stranded DNA. Once the long DNA molecules are hydrolyzed as a result of DNase I activity, dissociation of methyl green from the DNA occurs. The free methyl green decolorizes spontaneously, probably as a result of tautomerization of the dye.

For the evaluation of DNase I activity, tested DNase I variants were purified by dialysis against a formulation buffer (150 mM NaCl, 1 mM CaCl₂, pH 6.1-6.5). A standard curve was prepared by dilution of the standard, unmodified DNase I in an activity buffer (25 mM HEPES-NaOH, 4 mM CaCl₂, 4 mM MgCl₂, 0.1% bovine serum albumin, 0.05% TWEEN-20, pH 7.5) at concentrations ranging from 0.3 to 20 ng/mL at 2-fold series dilutions. Samples and controls were diluted in a similar manner. For pharmacokinetic analysis, standard curve and controls were spiked according to sample dilution.

100 μL of standards, controls and samples were added in duplicates to a 96-well plate (NUNC) containing 100 μL of DNA-methyl green substrate and the contents were mixed thoroughly. The plates were then incubated overnight at 37° C. and absorbance was then measured at a wavelength of 620 nm. Absorbance was plotted versus standard concentrations and the data were fit to a 4-parameter logistic model by the nonlinear regression method of Marquardt. Concentration of DNase and DNase variants was then calculated.

MALDI-TOF (Matrix-Assisted Laser Desorption/Ionization-Time of Flight) Mass Spectrometry:

Sample preparation: A matrix solution was prepared by mixing 375 μL of 20 mg/mL solution of 2,5-DHAP (2,5-dihydroxyacetophenone) in ethanol and 125 μL of 18 mg/mL DAC (diammonium hydrogen citrate) aqueous solution. 2 μL of sample solution were mixed with 2 μL of a 2% TFA solution, followed by 2 μL of the matrix solution. This ternary mixture was then pipetted up and down until crystallization began, whereby the previously transparent mixture becomes opaque. A volume of 0.5 μL of this mixture was applied on a MALDI steel target plate. After evaporation of the solvent, the target was inserted into the mass spectrometer.

Mass Spectrometry: MALDI-TOF mass spectra were acquired using a MALDI-TOF/TOF Autoflex™ speed mass spectrometer (Bruker Daltonilk GmbH). The mass spectrometer was equipped with a Smartbeam™-II solid-state laser (modified Nd:YAG laser) λ=355 nm, and was operated in a positive ion linear mode within a mass range from 20000 to 200000 m/z or from 60000 to 200000 m/z. Laser fluency was optimized for each sample. The laser was operated at a frequency of 2 kilohertz and spectra were accumulated in multiples of 1000 laser shots, with 2000 shots in total.

SDS-PAGE:

DNase I and modified DNase species were analyzed by SDS-PAGE. Detection of proteins was achieved by Coomassie brilliant blue staining (Bio-Rad) according to the manufacturer's instructions.

### Example 1

### Effect of PEG-Modification on Plant Recombinant Human DNase I (prh-DNase I) Activity

Plant recombinant human DNase I (prh-DNase I) was modified with PEG (5 kDa) according to procedures described in the Materials and Methods section hereinabove, using PEG-Ald (PEG-propionaldehyde) from 3 different suppliers and PEG-NHS (methoxy-PEG-N-succinimidyl active esters) from 2 different suppliers in a 1:200 (protein:PEG) molar ratio. The reaction mixture was then purified by dialysis (using a filter with a 30 kDa cutoff) to formulation buffer: comprising 1 mM CaCl₂ and 150 mM NaCl. The products were analyzed by SDS-PAGE, by optical density (OD) to determine protein content, and by methyl green-based assay to determine enzymatic activity, as described hereinabove.

As shown in FIG. 1, prh-DNase I has a molecular weight of about 32 kDa, and migrates to the corresponding place in SDS-PAGE; whereas the PEGylated prh-DNase I species exhibit a higher molecular weight, and when PEGylated by PEG-Ald (5000 Da), the main part of the bands is observed above the 95 kDa marker. The increment of 60 kDa in apparent molecular weight corresponds to modification by about 6 PEG moieties of 5 kDa each, as PEG migrates in SDS-PAGE at approximately twice of the degree corresponding to its molecular weight. As further shown therein, the modifications by PEG-NHS were less efficient and efficiency varied between PEG-NHS from two suppliers, probably due to different reagent quality. The majority of bands were below the 95 kDa marker. PEGylated DNase variants modified by 1-5 PEG moieties are observed.

As shown in Table 3, prh-DNase I modified by PEG-Ald maintained over 85% enzymatic activity; whereas the PEG-NHS-modified prh-DNase I (despite having a lower level of modification, as shown in FIG. 1) affected the enzymatic activity of the modified protein to a considerably greater extent, with only 23-27% activity being maintained.

TABLE 3
Protein content and enzymatic activity of prh-DNase
I PEGylated with 5000 Da PEG, the ratio between
content and activity representing % activity.

| | Content by | | | |
|---|---|---|---|---|
| | (OD₂₈₀) | Enzymatic Activity | | |
| prh-DNase I | Mean | Mean | SD | |
| modification | (mg/mL) | (mg/mL) | (mg/mL) | % Activity |
| mPEG-Ald | 1.09 | 1.02 | 0.07 | 93.5 |
| (5 kDa PEG) | 1.25 | 1.07 | 0.08 | 85.5 |
| | 1.07 | 0.96 | 0.09 | 89.7 |
| mPEG-NHS | 2.35 | 0.63 | 0.04 | 27.0 |
| (5 kDa PEG) | 1.48 | 0.34 | 0.00 | 23.2 |

As shown in Table 4, prh-DNase I modified by 2000 Da PEG-Ald maintained considerably greater enzymatic activity than did prh-DNase I modified by 2000 Da PEG-NHS, similarly to the abovementioned results obtained with 5000 Da PEG.

TABLE 4
Protein content and enzymatic activity of prh-DNase
I PEGylated with 2000 Da PEG, the ratio between
content and activity representing % activity.

| | Content | | | |
|---|---|---|---|---|
| | (OD₂₈₀) | Enzymatic Activity | | |
| prh-DNase I | Mean | Mean | SD | |
| modification | (mg/mL) | (mg/mL) | (mg/mL) | % Activity |
| mPEG-aldehyde | 1.55 | 1.43 | 0.03 | 92.2 |
| (2 kDa PEG) | | | | |
| mPEG-NHS | 1.76 | 0.34 | 0.04 | 19.6 |
| (2 kDa PEG) | | | | |

Without being bound by any particular theory, it is believed that the difference in activity is associated with the fact that amidation (with PEG-NHS) changes positively charged amine groups to neutral amide groups, whereas in reductive amination (with PEG-Ald), a positively charged amine group remains, which may facilitate interaction with the negatively charged substrate (DNA).

The effect of PEGylation conditions and degree of PEGylation on DNase I modification was assessed by reacting prh-DNase I according to procedures described hereinabove with different amounts of PEGylation reagent, namely, 200, 400 and 600 molar equivalents versus the protein. The products were analyzed by SDS-PAGE and MALDI-TOF mass spectrometry to determine changes in molecular weight upon modification.

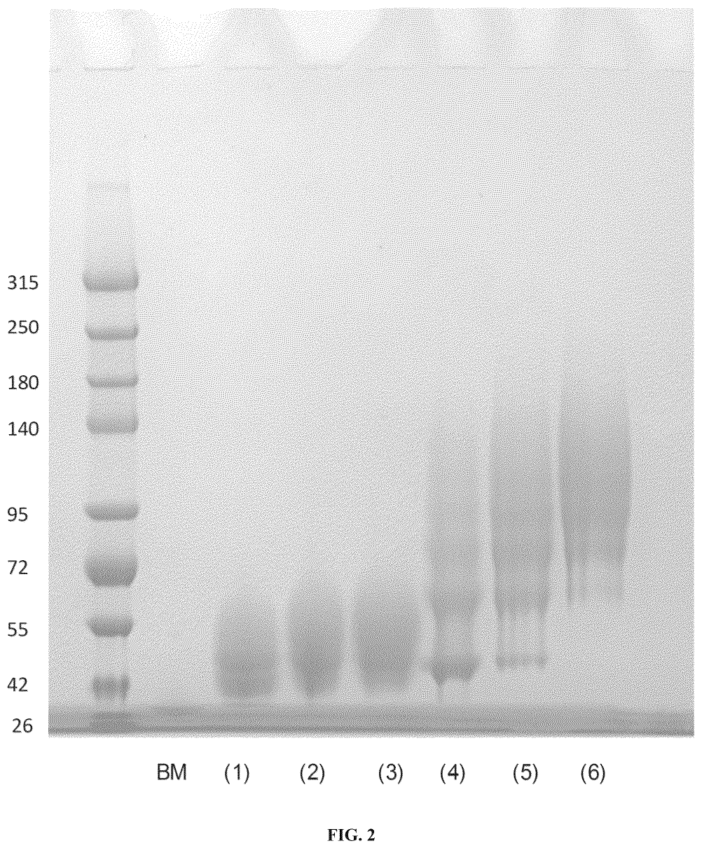

As shown in FIG. 2, prh-DNase I was modified with an average of 2, 3 and 4 PEG moieties upon reaction with 200, 400, and 600 molar equivalents, respectively, of 2000 Da PEG-Ald; and with an average of 4, 5, and 6 PEG moieties upon reaction with 200, 400, and 600 molar equivalents, respectively, of 5000 Da PEG-Ald (as determined by SDS-PAGE analysis).

The number of PEG moieties, as determined by MALDI-TOF mass spectrometry (data not shown), was similar to the number determined by SDS-PAGE analysis (as described hereinabove).

These results indicate that the degree of PEG modification of DNase correlated to the ratio of PEGylation reagent to DNase in a controllable manner.

### Example 2

### Pharmacokinetics of Exemplary PEGylated DNase I in Rats

Pulmozyme® recombinant human DNase is cleared rapidly from the systemic circulation following intravenous administration. A pharmacokinetic investigation of prh-DNase I in rats also demonstrated a short half-life of the enzyme (7.1 minutes, when 1 mg/kg body weight was injected intravenously; data not shown).

In order to assess the effect of PEGylation as described herein on DNase I, prh-DNase I was PEGylated by PEG-Ald (5000 Da PEG) according to procedures described hereinabove, using 200 molar equivalents of PEG-Ald, and purified using preparative size exclusion chromatography (SEC). As determined by an enzymatic activity assay, the PEGylated prh-DNase I retained about 63% of the initial activity of non-modified prh-DNase I (also referred to herein as “before modification” or as “prh-DNase I” per se).

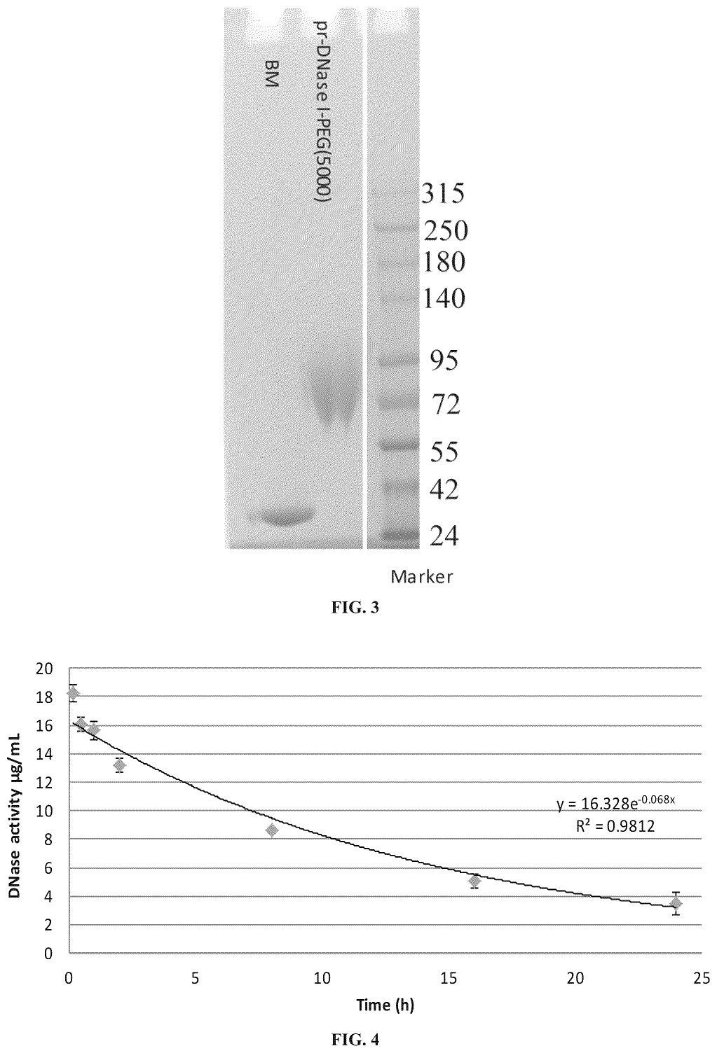

As shown in FIG. 3, the DNase was modified by 3-5 PEG chains, as determined by SDS-PAGE analysis.

Five Wistar rats were injected intravenously with the PEGylated DNase I at a dose of 1 mg/kg body weight (as quantified based on enzymatic activity). Blood samples were collected into heparin tubes 10 minutes and 0.5, 1, 2, 8, 16 and 24 hours after the IV injection, and the plasma fraction of blood was separated. Plasma samples were analyzed by methyl green based activity assay, according to procedures described hereinabove.

As shown in FIG. 4, DNase activity in blood declined gradually upon injection of PEGylated DNase I, with a half-life (associated with clearance) of about 10.2 hours.

These results indicate that modification of DNase as described herein considerably lengthens the duration of DNase activity in blood and reduces the rate of clearance.

In an additional study, the pharmacokinetics of 3 PEGylated prh-DNase variants (prepared according to procedures described hereinabove) and two non-PEGylated variants were compared, as follows:
Group A: non-modified DNase I (prh-DNase I);
Group B: alidornase alfa (non-PEGylated prh-DNase I modified by amidation with ethylene diamine, as described in International Patent Application Publication WO 2016/108244);
Group C: modified DNase I (prh-DNase I) with about 4 moieties per protein of 2 kDa PEG (average total mass of conjugated PEG of about 8 kDa, as determined by MALDI-TOF mass spectrometry), prepared using 400 equivalents of PEG-Ald (2 kDa PEG);
Group D: modified DNase I (prh-DNase I) with about 3 moieties per protein of 5 kDa PEG (average total mass of conjugated PEG being of 15 kDa, as determined by MALDI-TOF mass spectrometry), prepared using 100 equivalents of PEG-Ald (5 kDa PEG); and
Group E: modified DNase I (prh-DNase I) with about 4 moieties per protein of 5 kDa PEG (average total mass of conjugated PEG being of 20 kDa, as determined by MALDI-TOF mass spectrometry), prepared using 100 equivalents of PEG-Ald (5 kDa PEG).

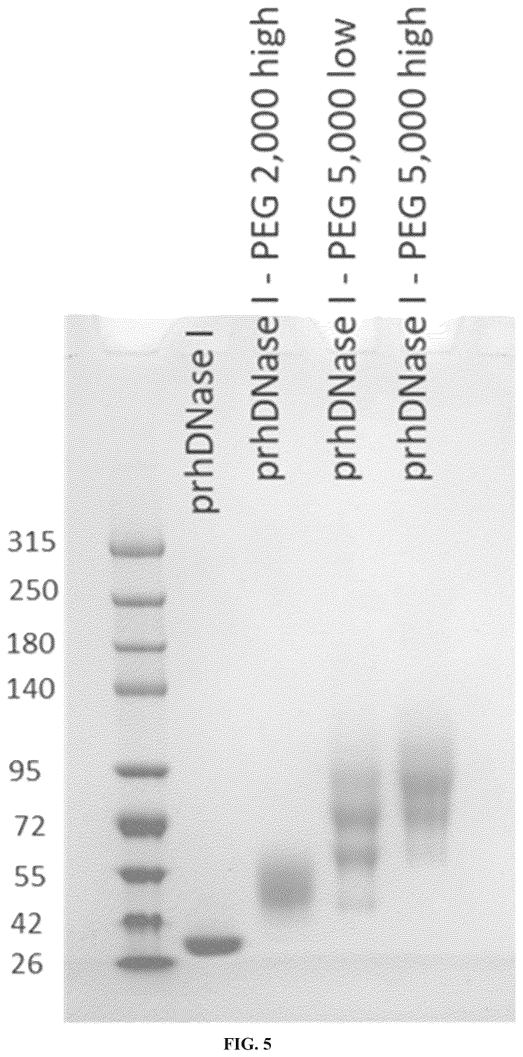

As shown in FIG. 5, the increase in mass, as determined by SDS-PAGE, for the three modified DNase I variants (Groups C, D and E) was consistent with the increase in mass determined by MALDI-TOF mass spectrometry, when considering that PEG is associated with an apparent mass in SDS-PAGE which is twice its real mass.

TABLE 5
Protein content and enzymatic activity (mean ±
standard deviation) of prh-DNase I PEGylated with various
molar equivalents of PEG (2 or 5 kDa), the ratio between
content and activity representing % activity

| | Enzymatic activity | Protein content | Activity |
|---|---|---|---|
| Sample | (mg/mL) | (mg/mL) | (%) |
| DNase I with about 4 | 1.77 ± 0.10 | 2.02 ± 0.01 | 87.9 |
| moieties of 2 kDa PEG | | | |
| DNase I with about 3 | 1.87 ± 0.10 | 2.04 ± 0.01 | 91.9 |
| moieties of 5 kDa PEG | | | |
| DNase I with about 4 | 1.47 ± 0.11 | 1.87 ± 0.00 | 78.9 |
| moieties of 5 kDa PEG | | | |

The DNase I variants were intravenously injected to Sprague Dawley rats (8 weeks old) at a dose of 1 mg/kg body weight (wherein concentration was determined based on activity, as shown in Table 5), with six animals for each test group. Blood samples were collected into lithium heparin tubes prior to injection and at different time intervals after IV injection and plasma was separated. The amount of active DNase in plasma samples was evaluated by methyl green-based activity assay.

As shown in FIGS. 6A-8, the PEGylated DNase I exhibited a considerably longer half-life (FIGS. 6A-7) and higher area under curve (AUC) (FIG. 8) than did non-PEGylated DNase I (non-modified DNase I or alidornase alfa), with the half-life and AUC being positively correlated to the number of PEG moieties and to the size of the PEG moieties.

These results indicate that PEGylation with multiple PEG moieties is highly effective at lengthening the activity of DNase I in vivo and that the DNase activity may be controlled by controlling the degree of PEGylation.

### Example 3

### Effect of Exemplary Long-Acting DNase on Sepsis in Cecal Ligation and Puncture Animal Model

DNase I with about 4 moieties of 5 kDa PEG as described in Example 2 (the exemplary modified DNase I with the longest half-life described therein) was selected for further study in a mouse model of sepsis induced in male C57BL/6 mice (8-9 weeks old) by cecal ligation and puncture (CLP).

The cecum was ligated about 1 cm below the end of the cecum and punctured twice, using an 18-gauge needle, and extruding about 1 cm of fecal matter. All animals received 1 mL of saline subcutaneously immediately following the surgery, as well as antibiotic therapy (subcutaneous ertapenem sodium, 30 mg/kg) 1 hour after surgery and thereafter every 12 hours, up to 48 hours. Liquid resuscitation (1 ml saline) was given 4 hours after surgery and thereafter with antibiotics.

The validity of the sepsis model was confirmed by determining that 24 hours after CLP, serum levels of urea, serum glutamic-oxaloacetic transaminase (SGOT, a.k.a. aspartate transaminase or AST), creatine phosphokinase and serum glutamic pyruvic transaminase (SGPT, a.k.a. alanine transaminase or ALT) (as determined by American Medical Laboratories central laboratory services), and serum levels of cell-free DNA (as determined using a Quant-iT™ PicoGreen™ double strand DNA assay kit (Invitrogen) according to manufacturer's instructions, with incubation with 1.8 mg/ml proteinase K for 30 minutes at 55° C. to reduce background signal), were considerably increased as compared to untreated control mice (data not shown).

The mice were injected intravenously with 10 mg/kg of the modified DNase I (or a control) 1, 4 or 8 hours after CLP, and the survival rate was determined every 12 hours; with 5 animals per test group. Saline and non-modified prhDNase I were each used as a control:

As shown in FIGS. 9A and 9B, administration of a single dose of DNase I (non-modified or modified) 1 or 4 hours after surgery enhanced survival as compared to saline, with modified DNase I enhancing survival to a greater extent than non-modified DNase I. As further shown therein, administration or modified DNase I after 4 hours after surgery enhanced survival to a greater extent than did administration 1 hour after surgery.

These results indicate that modified DNase as described herein provides an enhanced therapeutic effect against sepsis, even when antibiotics are also administered. These results further indicate that timing of DNase administration may have an important effect on the therapeutic effect, which may be due to the role of NETs in the early immune response to a septic insult [Mai et al., Shock 2015, 44:166-172].

The effect of modified DNase I was retested in the same model (using saline as a control) upon administration of the modified DNase I 4 and 8 hours after surgery.

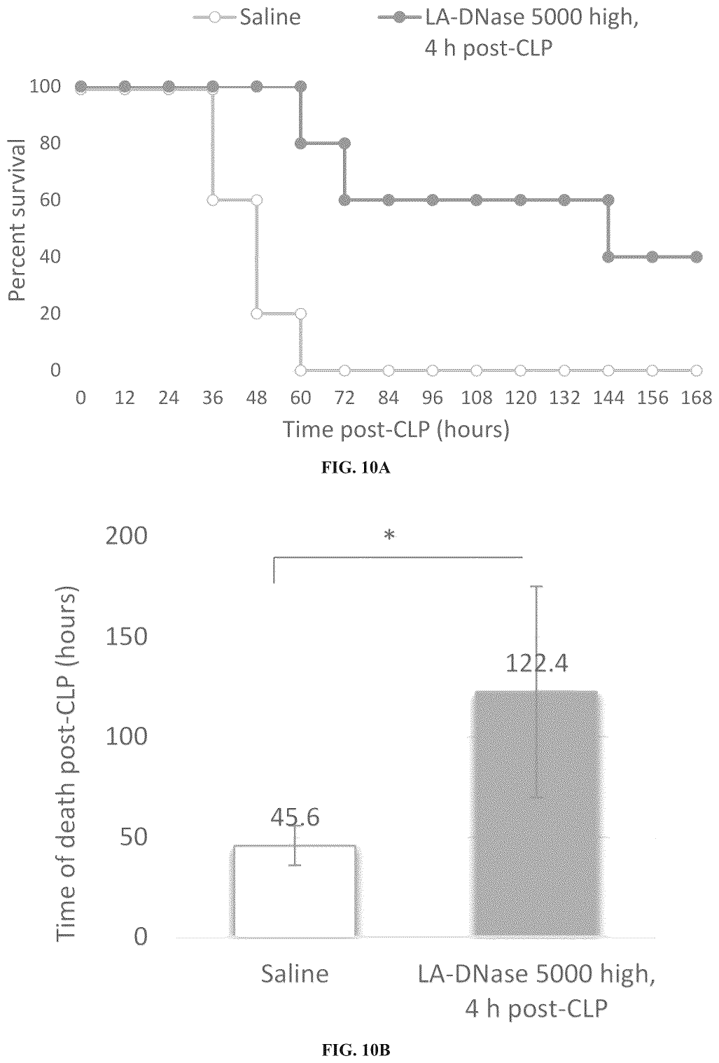

As shown in FIGS. 10A and 10B, administration of a single dose of modified DNase I 4 hours after surgery considerably enhanced survival as compared to saline.

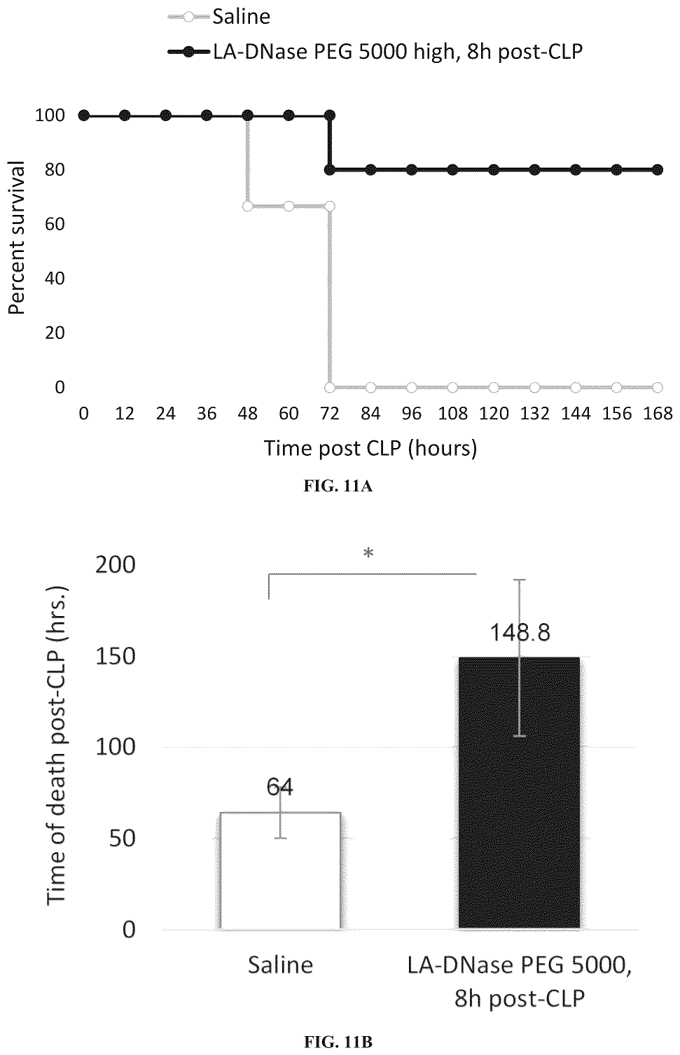

As shown in FIGS. 11A and 11B, administration of a single dose of modified DNase I 8 hours after surgery was highly effective at enhancing survival as compared to saline.

These results indicate that administration of DNase about 8 hours after sepsis induction is particularly effective for providing a therapeutic effect against sepsis.

In addition, the effect of dosage was assessed in a sepsis model, by administering doses of 10, 5, 1 and 0.1 mg/kg body weight of modified DNase I 4 hours after CLP (according to procedures described hereinabove).

As shown in FIGS. 12A and 12B, each of the tested doses of modified DNase I reduced mortality, but the reduction in mortality was dose-dependent, with no mortality being observed 7 days after CLP upon administration of the highest dose (10 mg/kg).

These results further confirm that modified DNase as described herein provides a therapeutic effect against sepsis.

### Example 4

### Additional Study of Exemplary Long-Acting DNase in Cecal Ligation and Puncture Sepsis Model

Sepsis is induced in mice by cecal ligation and puncture (CLP), followed by antibiotic treatment and administration (e.g., 4 hours after CLP) of modified DNase I (with non-modified DNase I and/or saline as a control), according to procedures described in Example 3.

Serum is then collected (e.g., 24 hours after CLP), and levels of organ damage biomarkers (e.g., creatine phosphokinase, urea, serum glutamic-oxaloacetic transaminase (SGOT), serum glutamic pyruvic transaminase (SGPT) and/or endocan), circulating cell-free DNA/NETs (e.g., using a Quant-iT™ PicoGreen™ double strand DNA assay kit as described in Example 3, and/or an ELISA assay based on anti-double stranded DNA antibodies), TNF, IL-6, and myeloperoxidase (MPO) in lung tissue (e.g., using an ELISA assay), and/or bacterial levels in the blood are optionally evaluated, using suitable techniques known in the art, in order to evaluate the ability of the modified DNase I to reduce cell-free DNA levels and/or attenuate organ damage.

### Example 5

### Effect of Exemplary Long-Acting DNase on Viral Infection

Mice are challenged with lethal doses of influenza virus (e.g., about 500 plaque forming units of virus).

Alternatively or additionally, influenza A virus A/Puerto Rico/8/34 H1N1 (PR8) obtained from the American Type Culture Collection (Manassas, VA) is propagated in embryonated eggs at 37° C. for 72 hours, and the allantoic fluid is harvested.

The effect of exemplary long-acting DNase (optionally administered at 1 mg/kg), prepared by PEGylation as described in any of the respective embodiments hereinabove (and any combination thereof), is compared with that of non-modified DNase at the same dosage (e.g., 1 mg/kg) and saline control. In particular, the effect of exemplary long-acting DNase on survival and/or on post-mortem BALF (bronchoalveolar lavage fluid) content of NETs (neutrophil extracellular traps)/DNA-related entities is assessed (e.g., as described in Narasarju et al. [Am J Pathol 2011, 179:199-210]).

Virus titers are optionally determined by the plaque assay via infection of Madin-Darby canine kidney (MDCK) cells, according to procedures such as described by Lin et al. [PLoS ONE 2017, 12:e0172299].

NETs (neutrophil extracellular traps) are optionally measured according to procedures such as described by de Buhr & von Kockritz-Blickwede [Detection, Visualization, and Quantification of Neutrophil Extracellular Traps (NETs) and NET Markers, pp. 425-442, in: Quinn M., Deteo F. (Us) Neutrophil. Methods in Molecular Biology, vol 2087. Humana, New York, NY].

The ability of the long-acting DNase (e.g., relative to non-modified DNase) to reduce mortality, virus titer, BALF content and/or NET content is evaluated.

### Example 6

### Effect of Exemplary Long-Acting DNase on Stroke

A patient having a stroke is treated with a single dose of an exemplary long-acting DNase, prepared as described hereinabove, along with tissue plasminogen activator (tPA) and/or other current standard practice of care.

The therapeutic window for use of tPA, secondary damage from the ischemia-reperfusion injury, and/or disability resulting from the stroke, are optionally evaluated, e.g., in comparison with tPA administered without the exemplary long-acting DNase.

Without being bound by any particular theory, it is believed that co-administration of exemplary long-acting DNase and tPA reduces the time needed for clot dissolution, and reduces the number of clots that are not dissolved by tPA, thereby reducing the need of endovascular procedures, and increasing the therapeutic window for use of tPA.

### Example 7

### Effect of Exemplary Long-Acting DNase on Myocardial Infarction

Wild-type C57BL6/J mice (e.g., 8 weeks old) are subjected to permanent ligation of the left descending coronary artery to induce myocardial infarction (MI), with sham operation serving as a control, according to procedures such as described by Michael et al. [Am J Physiol 1995, 269:H2147-H2154]. The treatment groups are optionally: exemplary long-acting DNase (intravenous injection, 1 mg/kg); non-modified prh-DNase (intravenous injection, 1 mg/kg); and saline. The treatment is performed once before the reperfusion.

Infarction area, left ventricular remodeling, inflammation markers (TNF-α and/or other pro-inflammatory cytokines) and/or plasma cell-free DNA are optionally evaluated, for example, in order to assess an ability of the exemplary long-acting DNase (e.g., relative to non-modified DNase and/or saline) to decrease infarction area, inflammation markers and/or plasma cell-free DNA, and/or to increase left ventricular remodeling.

Left ventricular remodeling is optionally evaluated according to procedures such as described in Vogel et al. [Basic Res Cardiol 2015, 110:15]. Cell-free DNA (cfDNA) is optionally collected and evaluated according to procedures such as described in Alborelli et al. [Cell Death Dis 2019, 10:534]. Inflammation markers are optionally evaluated using commercially available ELISA-based kits.

### Example 8

### Effect of Exemplary Long-Acting DNase in Lipopolysaccharide-Induced Sepsis Animal Model

Mice are divided into 4 groups (e.g., of 5 animals each) and treated as follows:
1) control (naive),
2) Lipopolysaccharide (LPS)+saline (LPS-induced endotoxic shock treated with saline subcutaneously),
3) Lipopolysaccharide (LPS)+prh-DNase I (LPS-induced endotoxic shock treated with prh-DNase I).
4) Lipopolysaccharide (LPS)+exemplary long-acting prh-DNase (LPS-induced endotoxic shock treated with exemplary long-acting DNase, prepared according to procedures such as described hereinabove).

The mice are treated with a sub-lethal dose of LPS and by saline or DNase (10 mg/kg, intravenous) 10 minutes before and 8 hours after endotoxic shock. Twelve hours after endotoxic shock induction, blood levels of organ damage biomarkers (e.g., creatine phosphokinase, blood urea nitrogen (BUN), and aspartate transaminase (AST)), circulating free DNA, TNF-α and myeloperoxidase (MPO) in lung tissue are optionally evaluated. Additionally, NET deposition in kidney tissues 12 hours after endotoxic shock induction is optionally evaluated.

Comparison of bio-marker levels between experimental groups can demonstrate the effect of PEGylation on the ability of the DNase to reduce an inflammatory reaction.

In order to assess an effect of DNase PEGylation on survival, the mice are treated as described hereinabove, except that a lethal dose of LPS is used, every 8 hours up to day 3.

### Example 9

### Effect of Exemplary Long-Acting DNase in Animal Model of Post-Chemotherapy Neutropenia

The effect of exemplary long-acting DNase, prepared according to procedures described hereinabove, on chemotherapy-induced neutropenia is evaluated according to procedures such as described by Mittra et al. [Annals of Oncology 2017, 28:2119-2127].

Briefly, a single injection of adriamycin (10 mg/kg) is followed by daily blood count for 10 days. Mice and/or rats are divided into 3 groups (e.g., of 5 animals each) and treated as follows:
1) adriamycin (10 mg/kg, intraperitoneal);
2) adriamycin (10 mg/kg, intraperitoneal)+prh-DNase (1 mg/kg, intravenous);
3) adriamycin (10 mg/kg, intraperitoneal)+exemplary long-acting prh-DNase (1 mg/kg, intravenous);
Blood count and inflammation biomarkers (e.g., TNF-α and other pro-inflammatory cytokines) are evaluated (e.g., as described hereinabove) in order to assess the ability of the exemplary long-acting DNase to improve blood counts and/or reduce inflammation biomarkers.

### Example 10

### Effect of Exemplary Long-Acting DNase in Animal Model of Inflammatory Bowel Disease (IBD) And Colitis

In order to investigate the effect of NET degradation and neutrophil depletion on the progression of colitis, an induced colitis mouse model is used according to procedures such as described in Li et al. [J Crohn's Colitis 2020, 14:240-253).

Mice (e.g., 8 weeks old, male) are fed 3% (w/v) dextran sulfate sodium (DSS, e.g., MW 36-40 kDa) in the drinking water for 5 days, followed by normal drinking water until day 8. The animals are weighed daily and monitored for signs of distress. The mice are divided into 3 groups (e.g., of 5 animals each) and treated as follows:
(1) DSS+saline;
(2) DSS+prh-DNase;
(3) DSS+exemplary long-acting prh-DNase;
DNase variants are given intravenously as a single dose of 5 mg/kg at day 5 in the model induction. NET formation and cell-free DNA levels are evaluated in addition to weight loss, disease activity index, level of colon shortening, and/or histological signs of inflammation. The increase of serum cell-free DNA and NET formation in DSS-induced colitis on the 4th and 6th day after DSS initiation is determined, as well as the ability of the exemplary long-acting DNase to reduce NET formation, cell-free DNA, weight loss, disease activity index, colon shortening, and/or histologic signs of inflammation, and/or to increase survival (e.g., in comparison with non-modified DNase).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A modified DNase protein comprising a DNase polypeptide attached to at least two poly(alkylene glycol) moieties.

2. The modified DNase protein of claim 1, wherein at least a portion, or each, of said at least two poly(alkylene glycol) moieties has a molecular weight of no more than about 10 kDa.

3. (canceled)

4. The modified DNase protein of claim 1, wherein said polypeptide is attached to from 2 to 7 poly(alkylene glycol) moieties.

5. The modified DNase protein of claim 1, wherein said polypeptide is attached to at least three poly(alkylene glycol) moieties.

6. (canceled)

7. The modified DNase protein of claim 1, wherein at least a portion, or each, of said poly(alkylene glycol) moieties are monofunctional poly(alkylene glycol) moieties.

8. The modified DNase protein of claim 1, wherein at least a portion, or each, of said poly(alkylene glycol) moieties comprise an alkylene group covalently attached to a nitrogen atom of an amine group in said polypeptide.

9. The modified DNase protein of claim 8, wherein said amine group is comprised by a lysine residue side chain and/or the N-terminus.

10. The modified DNase protein of claim 9, wherein at least % of the amine groups comprised by a lysine residue side chain and the N-terminus in said polypeptide are covalently attached to said poly(alkylene glycol) moieties.

11. The modified DNase protein of claim 1, wherein at least a portion, or each, of said poly(alkylene glycol) moieties have formula I:
-L₂-L₁-[O—(CH₂)m]n-O—R₁ Formula I
wherein:
L₁ and L₂ are each independently a hydrocarbon moiety or absent;
R₁ is hydrogen or a hydrocarbon moiety;
m is an integer in a range of from 2 to 10; and
n is an integer in a range of from 2 to 1000.

12. The modified DNase protein of claim 1, wherein at least a portion, or each, of said poly(alkylene glycol) moieties have formula I′:
—CH₂-L₁-[O—(CH₂)m]n-O—R₁ Formula I′
wherein:
L₁ is a hydrocarbon moiety or absent;
R₁ is hydrogen or a hydrocarbon moiety;
m is an integer in a range of from 2 to 10; and
n is an integer in a range of from 2 to 1000.

13. The modified DNase protein of claim 11, wherein n is in a range of from 20 to 200.

14. The modified DNase protein of claim 11, wherein L₁ is an unsubstituted alkylene.

15. The modified DNase protein of claim 12, wherein L₁ is an unsubstituted alkylene.

16. The modified DNase protein of claim 1, wherein at least a portion, or each, of said poly(alkylene glycol) moieties are polyethylene glycol moieties.

17. The modified DNase protein of claim 1, wherein said polypeptide is a recombinant polypeptide.

18. The modified DNase protein of claim 17, wherein said polypeptide is a plant recombinant polypeptide.

19. The modified DNase protein of claim 1, wherein said DNase protein is a DNase I protein.

20. The modified DNase protein of claim 19, wherein said DNase I protein has at least 80% homology to a human DNase I protein.

21. The modified DNase protein of claim 20, wherein the DNase I protein comprises or has the amino acid sequence as set forth in SEQ ID NO: 2.

22. The modified DNase protein of claim 20, wherein the DNase I protein comprises or has the amino acid sequence as set forth in SEQ ID NO: 1.

23. A pharmaceutical composition comprising the modified DNase protein of any one of claim 1 and a pharmaceutically acceptable carrier.

24. A method of treating a disease or disorder in which DNase activity is beneficial in a subject in need thereof, the method comprising administering to the subject the modified DNas protein of claim 1, thereby treating the disease or disorder.

25. The method of claim 24, wherein said disease or disorder is selected from the group consisting of thrombosis, vascular occlusion, an inflammatory disease or disorder, an autoimmune disease or disorder, a bronchopulmonary disease, a cardiovascular disease, a metabolic disease, a cancer, a neurodegenerative disease or disorder, a disease or disorder associated with an infection, liver damage, fibrosis, and a ductal occlusion.

26. The method of claim 24, wherein said disease or disorder is selected from the group consisting of acute coronary syndrome, acute kidney injury, acute lung injury, acute respiratory distress syndrome, allergies, Alzheimer's disease, amyotrophic lateral sclerosis, arthritis, asthma, atelectasis, atherosclerosis, atopic dermatitis, bipolar disorder, bronchiectasis, bronchiolitis, bronchitis and tracheobronchitis, cholangitis, chronic kidney disease, chronic neutrophilia, chronic obstructive pulmonary disease, chronic suppurative lung disease conjunctivitis, common cold, cystic fibrosis, deep vein thrombosis, diabetes, disseminated intravascular coagulation, dry eye disease, empyema, endocarditis, female infertility, gout, graft-versus-host disease, hematomas, hemothorax, heparin-induced thrombocytopenia, hepatorenal syndrome, Huntington's disease, inflammatory bowel disease, intrabiliary blood clots, ischemia-reperfusion injury, Kartegener's syndrome, leukemia, leukostasis, liver cirrhosis, lupus nephritis, male infertility, mastitis, myocardial infarction, neutropenia, neutrophil aggregation, obstruction of the vas deferens, pancreatitis, Parkinson's disease, pneumonia, post-pneumatic anemia, primary ciliary dyskinesia, psoriasis, rhabdomyolysis, sarcoidosis, schizophrenia, sepsis, sickle cell disease, sinusitis, Sjogren's syndrome, smoke-induced lung injury, solid tumors and/or tumor metastasis, stroke, surgical adhesions, surgical and/or traumatic tissue injury, systemic inflammatory response syndrome, systemic lupus erythematosus, systemic sclerosis, thrombotic microangiopathy, tissue damage associated with irradiation and/or chemotherapy treatment, transfusion-induced lung injury, tuberculosis, vasculitis, venous thromboembolism, a viral, bacterial, fungal and/or protozoal infection, and a wound or ulcer.

27. (canceled)

28. A method of treating a disease or disorder associated with excess extracellular DNA in a fluid, secretion or tissue of a subject in need thereof, the method comprising administering to the subject the modified DNas protein of claim 1, thereby treating the disease or disorder.

29. The method of claim 24, wherein said disease or disorder is associated with neutrophil extracellular traps (NETs).

30. A process of preparing the modified DNase protein of claim 1, the process comprising:
(a) contacting said polypeptide with an agent that comprises a poly(alkylene glycol) attached to an aldehyde group, to obtain a conjugate of said polypeptide and said agent; and
(b) contacting said conjugate with a reducing agent.

31. The process of claim 30, wherein said reducing agent is selected from the group consisting of a picoline borane complex and a cyanoborohydride.

32. The process of claim 30, wherein said agent has formula II:
HC(═O)-L₁-[O—(CH₂)m]n-O—R₁ Formula II
wherein:
L₁ is a hydrocarbon moiety;
R₁ is hydrogen or a hydrocarbon moiety;
m is an integer in a range of from 2 to 10; and
n is an integer in a range of from 2 to 1000.

33-34. (canceled)


SEQUENCE LISTING (9 sequences)

SEQ ID NO: 1 - protein, 260 aa, Homo sapiens
LKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 2 - protein, 261 aa, Artificial sequence
  Putative amino acid sequence of the purified, plant expressed rhDNase I
GLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK

SEQ ID NO: 3 - DNA, 99 bp, Artificial sequence
  Coding sequence of polypeptide of ABPI leader signal
atgattgtgctttctgtgggatctgcttcttcttctccaattgtggtggtgttctctgtggctcttcttcttttctacttctctgagacttctcttggc

SEQ ID NO: 4 - DNA, 849 bp, Homo sapiens
atgaggggcatgaagctgctgggggcgctgctggcactggcggccctactgcagggggccgtgtccctgaagatcgcagccttcaacatccagacatttggggagaccaagatgtccaatgccaccctcgtcagctacattgtgcagatcctgagccgctatgacatcgccctggtccaggaggtcagagacagccacctgactgccgtggggaagctgctggacaacctcaatcaggatgcaccagacacctatcactacgtggtcagtgagccactgggacggaacagctataaggagcgctacctgttcgtgtacaggcctgaccaggtgtctgcggtggacagctactactacgatgatggctgcgagccctgcgggaacgacaccttcaaccgagagccagccattgtcaggttcttctcccggttcacagaggtcagggagtttgccattgttcccctgcatgcggccccgggggacgcagtagccgagatcgacgctctctatgacgtctacctggatgtccaagagaaatggggcttggaggacgtcatgttgatgggcgacttcaatgcgggctgcagctatgtgagaccctcccagtggtcatccatccgcctgtggacaagccccaccttccagtggctgatccccgacagcgctgacaccacagctacacccacgcactgtgcctatgacaggatcgtggttgcagggatgctgctccgaggcgccgttgttcccgactcggctcttccctttaacttccaggctgcctatggcctgagtgaccaactggcccaagccatcagtgaccactatccagtggaggtgatgctgaagtga

SEQ ID NO: 5 - protein, 282 aa, Homo sapiens [sequence omitted]

SEQ ID NO: 6 - DNA, 786 bp, Artificial sequence
  Coding sequence of the recombinant DNase I without the leader sequence
cttaaaatcgctgctttcaacatccaaactttcggagagactaagatgtctaacgctactcttgtgtcctacatcgttcagattctctccagatacgatattgctcttgttcaggaagttagggattctcaccttactgctgtgggaaagcttcttgataacctcaatcaggatgctccagatacttaccactacgttgtgtctgaaccacttggaagaaactcctacaaagagcgttacctctttgtttaccgtccagatcaagtttctgctgtggattcctactactacgatgatggatgtgagccatgcggaaacgatactttcaatagagagccagctatcgttcgttttttcagtaggttcactgaagttcgtgagtttgctattgtgccacttcatgctgctccaggtgatgctgttgctgagattgatgctctctacgatgtgtaccttgatgttcaagagaagtggggattggaggatgttatgctcatgggagatttcaatgctggatgctcttatgttaggccatctcagtggtcatctattaggctttggacttccccaactttccaatggcttatcccagattccgctgatacaactgctactccaactcattgtgcttacgataggattgtggtggctggaatgcttcttagaggtgctgttgttccagattctgctctcccattcaatttccaagctgcttacggactttctgatcaacttgctcaggctatttctgatcactacccagttgaggtgatgttgaagtgatga

SEQ ID NO: 7 - DNA, 885 bp, Artificial sequence
  Coding sequence of rhDNase I (ABPI+DNase I)
atgattgtgctttctgtgggatctgcttcttcttctccaattgtggtggtgttctctgtggctcttcttcttttctacttctctgagacttctcttggccttaaaatcgctgctttcaacatccaaactttcggagagactaagatgtctaacgctactcttgtgtcctacatcgttcagattctctccagatacgatattgctcttgttcaggaagttagggattctcaccttactgctgtgggaaagcttcttgataacctcaatcaggatgctccagatacttaccactacgttgtgtctgaaccacttggaagaaactcctacaaagagcgttacctctttgtttaccgtccagatcaagtttctgctgtggattcctactactacgatgatggatgtgagccatgcggaaacgatactttcaatagagagccagctatcgttcgttttttcagtaggttcactgaagttcgtgagtttgctattgtgccacttcatgctgctccaggtgatgctgttgctgagattgatgctctctacgatgtgtaccttgatgttcaagagaagtggggattggaggatgttatgctcatgggagatttcaatgctggatgctcttatgttaggccatctcagtggtcatctattaggctttggacttccccaactttccaatggcttatcccagattccgctgatacaactgctactccaactcattgtgcttacgataggattgtggtggctggaatgcttcttagaggtgctgttgttccagattctgctctcccattcaatttccaagctgcttacggactttctgatcaacttgctcaggctatttctgatcactacccagttgaggtgatgttgaagtgatga

SEQ ID NO: 8 - protein, 33 aa, Artificial sequence
  Arabidopsis derived ABPI endoplasmic reticulum targeting signal peptide
MIVLSVGSASSSPIVVVFSVALLLFYFSETSLG

SEQ ID NO: 9 - protein, 293 aa, Artificial sequence
  Plant recombinant human DNase I
MIVLSVGSASSSPIVVVFSVALLLFYFSETSLGLKIAAFNIQTFGETKMSNATLVSYIVQILSRYDIALVQEVRDSHLTAVGKLLDNLNQDAPDTYHYVVSEPLGRNSYKERYLFVYRPDQVSAVDSYYYDDGCEPCGNDTFNREPAIVRFFSRFTEVREFAIVPLHAAPGDAVAEIDALYDVYLDVQEKWGLEDVMLMGDFNAGCSYVRPSQWSSIRLWTSPTFQWLIPDSADTTATPTHCAYDRIVVAGMLLRGAVVPDSALPFNFQAAYGLSDQLAQAISDHYPVEVMLK